SEQUENCE LISTING (14 sequences)

SEQ ID NO: 1 - DNA, 1730 bp, Talaromyces leycettanus
atggcgtccctcttctctttcaagatgtacaaggctgctctcgtcctgtcttctctcctggccgctacgcaggctcagcaggccggcactctcacgacggagacccatccgtccctgacatggcagcaatgctcggccggtggcagctgcaccacccagaacggcaaggtcgtcatcgatgcgaactggcgttgggtgcacagcacgagcggaagcaacaactgctacaccggcaatacctgggacgctaccctatgccctgacgatgtgacctgcgccgccaactgtgcgctggacggtgccgactactcgggcacctacggagtgaccaccagcggcaactccctccgcctcaacttcgtcacccaggcgtcacagaagaacgtcggctcccgtctttacctgatggagaatgacacaacctaccagatcttcaagctgctgaaccaggagttcacctttgatgtcgatgtgtccaacctgccgtaagtgacttaccatgaacccctgacgctatcttcttgttggctcccagctgactggccaattcaagctgcggcttgaacggtgctctctacctggtggccatggacgccgatggtggcatggccaagtaccccaccaacaaggctggtgccaagtacggtaccgggtactgcgactcccagtgtccccgcgacctcaagttcatcaatggcgaggccaacgtcgagggctggcagccgtcgtccaacgatcccaactctggcattggcaaccacggatcctgctgcgcggagatggatatctgggaggccaacagcatctccaatgctgtcactccccacccgtgcgacactcccggccaggtgatgtgcaccggtaacaactgcggtggcacatacagcactactcgctatgcgggcacttgcgatcccgacggctgcgacttcaacccctaccgcatgggcaaccacagcttctacggccctaaacagatcgtcgataccagctcgaagttcaccgtcgtgacgcagttcctcacggatgacggcacctccaccggcaccctctctgaaatccgccgcttctatgtccagaacggccaggtgatcccgaactcggtgtcgaccatcagtggcgtgagcggcaactccatcaccaccgagttctgcactgcccagaagcaggccttcggcgacacggacgacttctcaaagcacggcggcctgtccggcatgagcgctgccctctctcagggtatggttctggtcatgagtctgtgggatgatgtgagtttgatggacaaacatgcgcgttgacaaagagtcaagcagctgactgagatgttacagcacgccgccaacatgctctggctcgacagcacctacccgaccaacgcgacctcctccacccccggtgccgcccgtggaacctgcgacatctcgtccggtgtccctgcggatgtcgaatccaacgaccccaacgcctacgtggtctactcgaacatcaaggttggtcccatcggctcgaccttcagcagcagcggctctggatcttcttcctctagctccaccactaccacgaccaccgcttccccaaccaccacgacctcctccgcatcgagcaccggcactggagtggcacagcactggggccagtgtggtggacagggctggaccggccccacaacctgcgtcagcccttatacttgccaggagctgaacccttactactaccagtgtctgtaa

SEQ ID NO: 2 - protein, 532 aa, Talaromyces leycettanus
MASLFSFKMYKAALVLSSLLAATQAQQAGTLTTETHPSLTWQQCSAGGSCTTQNGKVVIDANWRWVHSTSGSNNCYTGNTWDATLCPDDVTCAANCALDGADYSGTYGVTTSGNSLRLNFVTQASQKNVGSRLYLMENDTTYQIFKLLNQEFTFDVDVSNLPCGLNGALYLVAMDADGGMAKYPTNKAGAKYGTGYCDSQCPRDLKFINGEANVEGWQPSSNDPNSGIGNHGSCCAEMDIWEANSISNAVTPHPCDTPGQVMCTGNNCGGTYSTTRYAGTCDPDGCDFNPYRMGNHSFYGPKQIVDTSSKFTVVTQFLTDDGTSTGTLSEIRRFYVQNGQVIPNSVSTISGVSGNSITTEFCTAQKQAFGDTDDFSKHGGLSGMSAALSQGMVLVMSLWDDHAANMLWLDSTYPTNATSSTPGAARGTCDISSGVPADVESNDPNAYVVYSNIKVGPIGSTFSSSGSGSSSSSSTTTTTTASPTTTTSSASSTGTGVAQHWGQCGGQGWTGPTTCVSPYTCQELNPYYYQCL

SEQ ID NO: 3 - DNA, 1898 bp, Talaromyces leycettanus
atgcggtctctcctggctcttgcccctaccctgctcgcgcctgttgttcaggctcagcaaaccatgtggggtcaatgtaagttcttttcactgcttaccatgtataatctttgatatcaagcatcatatctgactcacgttttaggcggtggtcagggctggaccggacctaccatctgtgtagcaggcgcgacatgcagcacacagaacccttgtaagtcgggccttcatcaaaacttcaacatcaccacctcgatggagcaggagttgacctgatctttacccttagggtatgcgcagtgcaccccagcacctaccgcgccgacgaccttgcaaacaacaactacgacgagctcgaaatcgtccacgaccacgagctcgaagtcgtccacgaccacaggtggaagtggcggtggaactacgacctcaacgtcagccaccatcaccgcggctccatctggtaacccatactccggataccagctctatgtgaaccaggaatactcgtccgaggtgtacgcgtctgctattccttcccttaccggcactctggtcgcgaaggcaagcgccgcggcagaggtgccatctttcctgtggctgtaagtttttttgaccttgaatgaacgccctgtcctctacgagtggccgcaggagctaattgagatgccaatgaacagggacactgcctccaaggtgccactgatgggcacttacttgcaggatatccaggcgaagaacgctgctggcgccaaccccccatatgccggtcaattcgtggtttacgacttgccggatcgtgattgcgctgcattggccagcaatggagagtactccattgctaacaatggtgttgccaactacaaggcttacatcgactccatccgcgcgcttcttgttcaatactcgaacgtccatgtcatccttgtgatcggtgagctattgcagtctcgctttaaagcatttgactagatcaatgtcgctaatggtacctaccgcacagagcccgacagcttggccaaccttgtcaccaacctgaatgttcagaagtgtgctaatgctcagagtgcttacctggagtgcatcaactatgccctcactcagttgaacctcaagaacgttgctatgtacatcgatgctggtgcgtgaaccttccctagtcagcccaaaataactgaaataaagagacggagtgtactgattgtcatgcaggtcatgctggatggctcggctggcccgccaaccttagcccggccgctcaactctttgcttccgtataccagaatgcaagctccccagctgccgttcgcggcctggcaaccaacgtggccaactataatgcctggtcgatcgccacttgcccatcttacacccaaggcgaccccaactgcgacgagcagaaatacatcaacgctctggctccattgcttcagcaacagggatggtcatcagttcactttatcaccgataccggtaagtctgcctgtcctgccaaccatgcgttcaagagcgttgcaatcctaaccatgctggtatcttccaggccgtaacggtgtccagcctaccaagcagaatgcctggggtgactggtgcaacgttatcggaaccggcttcggtgtccgtcccaccaccaacactggcgatccattggaggatgctttcgtctgggtcaagcctggtggtgagagtgatggtacttccaactccacttcgcctcgctacgacgcccactgcggttacagtgatgctcttcagcctgctcctgaggctggtacctggttcgaggtaagcttctgcatactgagatcgagaatcctgaaagggttaacctgctaatgcttcggtgtttgatataggcttactttgagcaactccttaccaacgccaacccctctttctaa

SEQ ID NO: 4 - protein, 464 aa, Talaromyces leycettanus
MRSLLALAPTLLAPVVQAQQTMWGQCGGQGWTGPTICVAGATCSTQNPWYAQCTPAPTAPTTLQTTTTTSSKSSTTTSSKSSTTTGGSGGGTTTSTSATITAAPSGNPYSGYQLYVNQEYSSEVYASAIPSLTGTLVAKASAAAEVPSFLWLDTASKVPLMGTYLQDIQAKNAAGANPPYAGQFVVYDLPDRDCAALASNGEYSIANNGVANYKAYIDSIRALLVQYSNVHVILVIEPDSLANLVTNLNVQKCANAQSAYLECINYALTQLNLKNVAMYIDAGHAGWLGWPANLSPAAQLFASVYQNASSPAAVRGLATNVANYNAWSIATCPSYTQGDPNCDEQKYINALAPLLQQQGWSSVHFITDTGRNGVQPTKQNAWGDWCNVIGTGFGVRPTTNTGDPLEDAFVWVKPGGESDGTSNSTSPRYDAHCGYSDALQPAPEAGTWFEAYFEQLLTNANPSF

SEQ ID NO: 5 - DNA, 835 bp, Penicillium sp.
atgctgtcttcgacgactcgcaccctcgcctttacaggccttgcgggccttctgtccgctcccctggtcaaggcccatggctttgtccagggcattgtcatcggtgaccaattgtaagtccctctcttgcagttctgtcgattaactgctggactgcttgcttgactccctgctgactcccaacagctacagcgggtacatcgtcaactcgttcccctacgaatccaacccaccccccgtcatcggctgggccacgaccgccaccgacctgggcttcgtcgacggcacaggataccaaggcccggacatcatctgccaccggaatgcgacgcccgcgccgctgacagcccccgtggccgccggcggcaccgtcgagctgcagtggacgccgtggccggacagccaccacggacccgtcatcacctacctggcgccgtgcaacggcaactgctcgaccgtcgacaagacgacgctggagttcttcaagatcgaccagcagggcctgatcgacgacacgagcccgccgggcacctgggcgtcggacaacctcatcgccaacaacaatagctggaccgtcaccattcccaacagcgtcgcccccggcaactacgtcctgcgccacgagatcatcgccctgcactcggccaacaacaaggacggcgcccagaactacccccagtgcatcaacatcgaggtcacgggcggcggctccgacgcgcctgagggtactctgggcgaggatctctaccatgacaccgacccgggcattctggtcgacatttacgagcccattgcgacgtataccattccggggccgcctgagccgacgttctag

SEQ ID NO: 6 - protein, 253 aa, Penicillium sp.
MLSSTTRTLAFTGLAGLLSAPLVKAHGFVQGIVIGDQFYSGYIVNSFPYESNPPPVIGWATTATDLGFVDGTGYQGPDIICHRNATPAPLTAPVAAGGTVELQWTPWPDSHHGPVITYLAPCNGNCSTVDKTTLEFFKIDQQGLIDDTSPPGTWASDNLIANNNSWTVTIPNSVAPGNYVLRHEIIALHSANNKDGAQNYPQCINIEVTGGGSDAPEGTLGEDLYHDTDPGILVDIYEPIATYTIPGPPEPTF

SEQ ID NO: 7 - DNA, 2502 bp, Thermoascus aurantiacus
atgcgcgcaattggacttctgccaggcatcatcggcattgctggtgctgcctgtccttacatgacaggcgagctgccgcgctccttcgccgagaaccctcatgctatcaaccgtcgtgctgagggtggtggtggtgccgctgccgagacggagaagttcctgtctcagttctacctgaacgacaacgacaccttcatgaccaccgatgttggcggtccaattgaggatcagaacagtctcagcgctggtgacagaggtcctaccctgctggaggacttcatcctccgtcaaaagatccagcgctttgaccatgagcgggtaggttgatctttactttcggccttcttcgagcggggtgatattaaaacaggtaataggtgcccgagcgtgctgtccatgcccgaggagcgggagcgcatggcgtgttcacatcctacgcagactggtccaacatcactgccgcttccttcctgtctgctgcaggaaaggagacacctgtctttgtccggttctccactgtagcaggaagcagaggaagcgcagacacggcgcgtgacgtgcacggtttcgcgacgaggttctacacggatgaagggaacttcggtaggcaactatcatgctctctttaaatgttctcgatctgacagccagcagacattgtcggcaacaacatccctgtcttcttcattcaagatgcgatccagttccccgacctgatccatgctgtcaagcccagcccgaacaacgagatccctcaggccgcaaccgcccatgactctgcctgggactttttcagccagcagccgagctctttgcatactctgttctgggctatggccggtcatggcattcctcgttcctacaggaacatggatggcttcggcatccacaccttccgctttgtgacggacgatggagcttccaagctcgtcaagttccactggacgtcgctgcagggcaaggcgagccttgtgtgggaagaggcacaggccgtggctggaaagaacgcggactatcaccgccaggacttgtgggacgcaatcgaggctggaaggtaccctgagtgggaggtaggctctccctgctatgtatggatgtgccagaagcttaataatggcctagctcggcgtgcaaatcatggatgaggaagaccagctgcgctttggcttcgatctgttggacccgaccaagatcgttcccgaggaatacgtgcccatcacgaagctcggaaagatgcagctcaaccgcaacccgctgaactacttcgccgagactgaacagatcatggtcagttcgccaccgtgttcggttgctcgttgctgaagtgctaacttgcaacagttccaaccgggtcacgttgtccgtggcattgatttcaccgaggaccctctgctccagggacgtctcttctcttacctcgacacccagctcaaccgccacggaggtccgaacttcgagcagatccccatcaaccggccacgcactccaattcacaacaacaaccgtgacggagccggtatgctagcccatgtattcctttctttatgcatttttatatgatgcgttctaacggcaacagcgcaaatgtacatccccctgaacaaggcggcgtacacccccaacactctgaacaacggctcccccaagcaggccaaccagacggtcggaaagggcttcttcacgactccaggccggacggcaagcggcaggcttgtgcgcgccgtcagctcaaccttcgccgacgtctggtcgcagcctcgtctgttctacaactccctcgtgccggcggagcagcagttcctgatcaacgcgatccgctttgagacggcccacatcacgagcgacgtcgtgaagaacaacgtcatcatccagctgaaccgcgtgagcaacaacctcgccaagagagtcgcccgggccatcggtgtcgcggagcccgagccagacccaaccttgtaccacaacaacaagaccgccaacgtcggggtgttcggcaagccgctcgccagactcgacggcctgcaggtcggggtcctcgccaccgtcaacaagcccgactcgatcaagcaggccgccagcctgaaggccagcttcgcggcggacaacgtcgacgtcaaggtcgtcgcggagcgcctcgccgacggcgtcgacgagacctactcggccgccgacgcggtcaacttcgacgccatcctggtcgccaacggcgctgagggcctcttcgcgcgcgacagcttcaccgccaggccggccaactcgaccaccgcgacgctctaccccgcgggccgcccgctccagatcctggtcgacgggttccgctacggcaagccggtcggggcgctcggcagcggcgccaaggcgctcgacgcagcggagatttcgacgacccgggccggcgtgtacgtcgccaactcgacgaccgacagcttcatcaatggcgtcagggacggtctgcggacgttcaagttcctggaccggttcgcgattgacgaggatgctgagtga

SEQ ID NO: 8 - protein, 740 aa, Thermoascus aurantiacus
MRAIGLLPGIIGIAGAACPYMTGELPRSFAENPHAINRRAEGGGGAAAETEKFLSQFYLNDNDTFMTTDVGGPIEDQNSLSAGDRGPTLLEDFILRQKIQRFDHERVPERAVHARGAGAHGVFTSYADWSNITAASFLSAAGKETPVFVRFSTVAGSRGSADTARDVHGFATRFYTDEGNFDIVGNNIPVFFIQDAIQFPDLIHAVKPSPNNEIPQAATAHDSAWDFFSQQPSSLHTLFWAMAGHGIPRSYRNMDGFGIHTFRFVTDDGASKLVKFHWTSLQGKASLVWEEAQAVAGKNADYHRQDLWDAIEAGRYPEWELGVQIMDEEDQLRFGFDLLDPTKIVPEEYVPITKLGKMQLNRNPLNYFAETEQIMFQPGHVVRGIDFTEDPLLQGRLFSYLDTQLNRHGGPNFEQIPINRPRTPIHNNNRDGAAQMYIPLNKAAYTPNTLNNGSPKQANQTVGKGFFTTPGRTASGRLVRAVSSTFADVWSQPRLFYNSLVPAEQQFLINAIRFETAHITSDVVKNNVIIQLNRVSNNLAKRVARAIGVAEPEPDPTLYHNNKTANVGVFGKPLARLDGLQVGVLATVNKPDSIKQAASLKASFAADNVDVKVVAERLADGVDETYSAADAVNFDAILVANGAEGLFARDSFTARPANSTTATLYPAGRPLQILVDGFRYGKPVGALGSGAKALDAAEISTTRAGVYVANSTTDSFINGVRDGLRTFKFLDRFAIDEDAE

SEQ ID NO: 9 - DNA, 3060 bp, Aspergillus fumigatus
atgagattcggttggctcgaggtggccgctctgacggccgcttctgtagccaatgcccaggtttgtgatgctttcccgtcattgtttcggatatagttgacaatagtcatggaaataatcaggaattggctttctctccaccattctacccttcgccttgggctgatggccagggagagtgggcagatgcccatcgacgcgccgtcgagatcgtttctcagatgacactggcggagaaggttaaccttacaacgggtactgggtgggttgcgacttttttgttgacagtgagctttcttcactgaccatctacacagatgggaaatggaccgatgcgtcggtcaaaccggcagcgttcccaggtaagcttgcaattctgcaacaacgtgcaagtgtagttgctaaaacgcggtggtgcagacttggtatcaactggggtctttgtggccaggattcccctttgggtatccgtgactgtgagctatacccgcggagtctttcagtccttgtattatgtgctgatgattgtctctgtatagctgacctcaactccgccttccctgctggtactaatgtcgccgcgacatgggacaagacactcgcctaccttcgtggcaaggccatgggtgaggaattcaacgacaagggcgtggacattttgctggggcctgctgctggtcctctcggcaaatacccggacggcggcagaatctgggaaggcttctctcctgatccggttctcactggtgtacttttcgccgaaactatcaagggtatccaagacgcgggtgtgattgctactgccaagcattacattctgaatgaacaggagcatttccgacaggttggcgaggcccagggatatggttacaacatcacggagacgatcagctccaacgtggatgacaagaccatgcacgagttgtacctttggtgagtagttgacactgcaaatgaggaccttgattgatttgactgacctggaatgcaggccctttgcagatgctgtgcgcggtaagattttccgtagacttgacctcgcgacgaagaaatcgctgacgaaccatcgtagctggcgttggcgctgtcatgtgttcctacaatcaaatcaacaacagctacggttgtcaaaacagtcaaactctcaacaagctcctcaaggctgagctgggcttccaaggcttcgtcatgagtgactggggcgctcaccacagcggtgtcggcgctgccctcgctgggttggatatgtcgatgcctggagacatttccttcgacgacggactctccttctggggcacgaacctaactgtcagtgttcttaacggcaccgttccagcctggcgtgtcgatgacatggctgttcgtatcatgaccgcgtactacaaggttggtcgtgaccgtcttcgtattccccctaacttcagctcctggacccgggatgagtacggctgggagcattctgctgtctccgagggagcctggaccaaggtgaacgacttcgtcaatgtgcagcgcagtcactctcagatcatccgtgagattggtgccgctagtacagtgctcttgaagaacacgggtgctcttcctttgaccggcaaggaggttaaagtgggtgttctcggtgaagacgctggttccaacccgtggggtgctaacggctgccccgaccgcggctgtgataacggcactcttgctatggcctggggtagtggtactgccgagttcccttaccttgtcacccccgagcaggctatccagcgagaggtcatcagcaacggcggcaatgtctttgctgtgactgataacggggctctcagccagatggcagatgttgcatctcaatccaggtgagtgcgggctcttagaaaaagaacgttctctgaatgaagttttttaaccattgcgaacagcgtgtctttggtgtttgtcaacgccgactctggagagggttacatcagtgtcgacggcaacgagggtgaccgcaaaaatctcactctgtggaagaacggcgaggccgtcattgacactgttgtcagccactgcaacaacacgattgtggttattcacagtgttgggcccgtcttgatcgaccggtggtatgataaccccaacgtcactgccatcatctgggccggcttgcccggtcaggagagtggcaactccctggtcgacgtgctctatggccgcgtcaaccccagcgccaagaccccgttcacctggggcaagactcgggagtcttacggggctcccttgctcaccgagcctaacaatggcaatggtgctccccaggatgatttcaacgagggcgtcttcattgactaccgtcactttgacaagcgcaatgagacccccatttatgagtttggccatggcttgagctacaccacctttggttactctcaccttcgggttcaggccctcaatagttcgagttcggcatatgtcccgactagcggagagaccaagcctgcgccaacctatggtgagatcggtagtgccgccgactacctgtatcccgagggtctcaaaagaattaccaagtttatttacccttggctcaactcgaccgacctcgaggattcttctgacgacccgaactacggctgggaggactcggagtacattcccgaaggcgctagggatgggtctcctcaacccctcctgaaggctggcggcgctcctggtggtaaccctaccctttatcaggatcttgttagggtgtcggccaccataaccaacactggtaacgtcgccggttatgaagtccctcaattggtgagtgacccgcatgttccttgcgttgcaatttggctaactcgcttctagtatgtttcactgggcggaccgaacgagcctcgggtcgttctgcgcaagttcgaccgaatcttcctggctcctggggagcaaaaggtttggaccacgactcttaaccgtcgtgatctcgccaattgggatgtggaggctcaggactgggtcatcacaaagtaccccaagaaagtgcacgtcggcagctcctcgcgtaagctgcctctgagagcgcctctgccccgtgtctactag

SEQ ID NO: 10 - protein, 863 aa, Aspergillus fumigatus
MRFGWLEVAALTAASVANAQELAFSPPFYPSPWADGQGEWADAHRRAVEIVSQMTLAEKVNLTTGTGWEMDRCVGQTGSVPRLGINWGLCGQDSPLGIRDSDLNSAFPAGTNVAATWDKTLAYLRGKAMGEEFNDKGVDILLGPAAGPLGKYPDGGRIWEGFSPDPVLTGVLFAETIKGIQDAGVIATAKHYILNEQEHFRQVGEAQGYGYNITETISSNVDDKTMHELYLWPFADAVRAGVGAVMCSYNQINNSYGCQNSQTLNKLLKAELGFQGFVMSDWGAHHSGVGAALAGLDMSMPGDISFDDGLSFWGTNLTVSVLNGTVPAWRVDDMAVRIMTAYYKVGRDRLRIPPNFSSWTRDEYGWEHSAVSEGAWTKVNDFVNVQRSHSQIIREIGAASTVLLKNTGALPLTGKEVKVGVLGEDAGSNPWGANGCPDRGCDNGTLAMAWGSGTAEFPYLVTPEQAIQREVISNGGNVFAVTDNGALSQMADVASQSSVSLVFVNADSGEGYISVDGNEGDRKNLTLWKNGEAVIDTVVSHCNNTIVVIHSVGPVLIDRWYDNPNVTAIIWAGLPGQESGNSLVDVLYGRVNPSAKTPFTWGKTRESYGAPLLTEPNNGNGAPQDDFNEGVFIDYRHFDKRNETPIYEFGHGLSYTTFGYSHLRVQALNSSSSAYVPTSGETKPAPTYGEIGSAADYLYPEGLKRITKFIYPWLNSTDLEDSSDDPNYGWEDSEYIPEGARDGSPQPLLKAGGAPGGNPTLYQDLVRVSATITNTGNVAGYEVPQLYVSLGGPNEPRVVLRKFDRIFLAPGEQKVWTTTLNRRDLANWDVEAQDWVITKYPKKVHVGSSSRKLPLRAPLPRVY

SEQ ID NO: 11 - DNA, 1520 bp, Talaromyces leycettanus
atggtccatctttcttccctggccctggctttggccgccggctcgcagctgtatgtgatccatgccatgactcgagaagtgctcccaaaactgactccaagtctcaatcttagtgcccaagctgcaggtcttaacactgctgccaaagcgattggaaagctctatttcggtaccgcaaccgacaacccggagctgtccgacagcacatacatgcaggagacggataacaccgatgatttcggccaactcaccccagctaactccatgaaggttcgctgacatcttagttcccccccccttttgggaatctgcgcggagatatgctgagccttcaaaactagtgggatgccaccgagccctctcagaacaccttcaccttcaccaacggtgatcagatcgcaaaccttgctaagagcaacggtcagatgctgagatgccacaacctggtgtggtacaaccagttgcccagctggggtaagcaaccggttctgttaatatcatcagcgtgaccgcatcgatcgtattgcgcggagattggaaagatttgcaagctaatgtcactacagtcaccagcggatcttggaccaatgccacgcttcttgcggccatgaagaaccacatcaccaacgttgtgacccactacaagggacagtgctacgcttgggatgttgtcaacgaaggtacgtttcgattcggcttccctcggaccgtatctgcaggcaaaaaggtcaatcaattgacaatcgtgatccccagctctcaacgatgatggcacctaccgatccaatgtcttctatcagtacatcggcgaggcatacattcccattgcctttgcgaccgctgccgccgccgatccaaacgcgaagctctactacaacgactacaacattgagtaccccggcgccaaggccaccgccgcccagaacatcgtcaagatggtcaaggcttacggcgcgaaaatcgacggtgtcggtctgcaatctcacttcatcgttggcagcacccctagccagagctcccagcagagcaacatggctgctttcaccgcgctcggcgtcgaggtcgccatcaccgaactggatatccgcatgacgttgccttccaccagtgctctcttggcccagcaatccaccgattaccagagcactgtgtcggcttgcgtgaacactccgaagtgcattggtatcaccctctgggactggaccgacaagtactcctgggttcccaacaccttctccggccaaggtgacgcctgcccctgggattctaactaccagaagaagcctgcctactacggtatcttgactgcgctcggaggcagcgcttccacctccaccaccaccactctggtgacctccaccaggacttcgactacgaccagcacttcggccacctccacgtctactggcgttgctcagcactggggccagtgcggtggtatcggctggacagggccgactacctgcgctagcccctacacctgccaggaactgaatccctactactaccagtgcctgtaa

SEQ ID NO: 12 - protein, 405 aa, Talaromyces leycettanus
MVHLSSLALALAAGSQLAQAAGLNTAAKAIGKLYFGTATDNPELSDSTYMQETDNTDDFGQLTPANSMKWDATEPSQNTFTFTNGDQIANLAKSNGQMLRCHNLVWYNQLPSWVTSGSWTNATLLAAMKNHITNVVTHYKGQCYAWDVVNEALNDDGTYRSNVFYQYIGEAYIPIAFATAAAADPNAKLYYNDYNIEYPGAKATAAQNIVKMVKAYGAKIDGVGLQSHFIVGSTPSQSSQQSNMAAFTALGVEVAITELDIRMTLPSTSALLAQQSTDYQSTVSACVNTPKCIGITLWDWTDKYSWVPNTFSGQGDACPWDSNYQKKPAYYGILTALGGSASTSTTTTLVTSTRTSTTTSTSATSTSTGVAQHWGQCGGIGWTGPTTCASPYTCQELNPYYYQCL

SEQ ID NO: 13 - DNA, 2391 bp, Talaromyces emersonii
atgatgactcccacggcgattctcaccgcagtggcggcgctcctgcccaccgcgacatgggcacaggataaccaaacctatgccaattactcgtcgcagtctcagccggacctgtttccccggaccgtcgcgaccatcgacctgtccttccccgactgtgagaatggcccgctcagcacgaacctggtgtgcaacaaatcggccgatccctgggcccgagctgaggccctcatctcgctctttaccctcgaagagctgattaacaacacccagaacaccgctcctggcgtgccccgtttgggtctgccccagtatcaggtgtggaatgaagctctgcacggactggaccgcgccaatttctcccattcgggcgaatacagctgggccacgtccttccccatgcccatcctgtcgatggcgtccttcaaccggaccctcatcaaccagattgcctccatcattgcaacgcaagcccgtgccttcaacaacgccggccgttacggccttgacagctatgcgcccaacatcaatggcttccgcagtcccctctggggccgtggacaggagacgcctggtgaggatgcgttcttcttgagttccacctatgcgtacgagtacatcacaggcctgcagggcggtgtcgacccagagcatgtcaagatcgtcgcgacggcgaagcacttcgccggctatgatctggagaactggggcaacgtctctcggctggggttcaatgctatcatcacgcagcaggatctctccgagtactacacccctcagttcctggcgtctgctcgatacgccaagacgcgcagcatcatgtgctcctacaatgcagtgaatggagtcccaagctgtgccaactccttcttcctccagacgcttctccgagaaaactttgacttcgttgacgacgggtacgtctcgtcggattgcgacgccgtctacaacgtcttcaacccacacggttacgcccttaaccagtcgggagccgctgcggactcgctcctagcaggtaccgatatcgactgtggtcagaccttgccgtggcacctgaatgagtccttcgtagaaggatacgtctcccgcggtgatatcgagaaatccctcacccgtctctactcaaacctggtgcgtctcggctactttgacggcaacaacagcgagtaccgcaacctcaactggaacgacgtcgtgactacggacgcctggaacatctcgtacgaggccgcggtggaaggtatcaccctgctcaagaacgacggaacgctgccgctgtccaagaaggtccgcagcattgcgctcatcggtccttgggccaatgccacggtgcagatgcagggtaactactatggaacgccaccgtatctgatcagtccgctggaagccgccaaggccagtgggttcacggtcaactatgcattcggtaccaacatctcgaccgattctacccagtggttcgcggaagccatcgcggcggcgaagaagtcggacgtgatcatctacgccggtggtattgacaacacgatcgaggcagagggacaggaccgcacggatctcaagtggccggggaaccagctggatctgatcgagcagctcagccaggtgggcaagcccttggtcgtcctgcagatgggcggtggccaggtggattcgtcgtcactcaaggccaacaagaatgtcaacgctctggtgtggggtggctatcccggacagtcgggtggtgcggccctgtttgacatccttacgggcaagcgtgcgccggccggtcgtctggtgagcacgcagtacccggccgagtatgcgacgcagttcccggccaacgacatgaacctgcgtccgaacggcagcaacccgggacagacatacatctggtacacgggcacgcccgtgtatgagttcggccacggtctgttctacacggagttccaggagtcggctgcggcgggcacgaacaagacgtcgactttcgacattctggaccttttctccacccctcatccgggatacgagtacatcgagcaggttccgttcatcaacgtgactgtggacgtgaagaacgtcggccacacgccatcgccgtacacgggtctgttgttcgcgaacacgacagccgggcccaagccgtacccgaacaaatggctcgtcgggttcgactggctgccgacgatccagccgggcgagactgccaagttgacgatcccggtgccgttgggcgcgattgcgtgggcggacgagaacggcaacaaggtggtcttcccgggcaactacgaattggcactgaacaatgagcgatcggtagtggtgtcgttcacgctgacgggcgatgcggcgactctagagaaatggcctttgtgggagcaggcggttccgggggtgctgcagcaataa

SEQ ID NO: 14 - protein, 796 aa, Talaromyces emersonii
MMTPTAILTAVAALLPTATWAQDNQTYANYSSQSQPDLFPRTVATIDLSFPDCENGPLSTNLVCNKSADPWARAEALISLFTLEELINNTQNTAPGVPRLGLPQYQVWNEALHGLDRANFSHSGEYSWATSFPMPILSMASFNRTLINQIASIIATQARAFNNAGRYGLDSYAPNINGFRSPLWGRGQETPGEDAFFLSSTYAYEYITGLQGGVDPEHVKIVATAKHFAGYDLENWGNVSRLGFNAIITQQDLSEYYTPQFLASARYAKTRSIMCSYNAVNGVPSCANSFFLQTLLRENFDFVDDGYVSSDCDAVYNVFNPHGYALNQSGAAADSLLAGTDIDCGQTLPWHLNESFVEGYVSRGDIEKSLTRLYSNLVRLGYFDGNNSEYRNLNWNDVVTTDAWNISYEAAVEGITLLKNDGTLPLSKKVRSIALIGPWANATVQMQGNYYGTPPYLISPLEAAKASGFTVNYAFGTNISTDSTQWFAEAIAAAKKSDVIIYAGGIDNTIEAEGQDRTDLKWPGNQLDLIEQLSQVGKPLVVLQMGGGQVDSSSLKANKNVNALVWGGYPGQSGGAALFDILTGKRAPAGRLVSTQYPAEYATQFPANDMNLRPNGSNPGQTYIWYTGTPVYEFGHGLFYTEFQESAAAGTNKTSTFDILDLFSTPHPGYEYIEQVPFINVTVDVKNVGHTPSPYTGLLFANTTAGPKPYPNKWLVGFDWLPTIQPGETAKLTIPVPLGAIAWADENGNKVVFPGNYELALNNERSVVVSFTLTGDAATLEKWPLWEQAVPGVLQQ

(19) United States
(12) Patent Application Publication
(10) Pub. No.: US 2018/0202011 A1
(43) Pub. Date: Jul. 19, 2018

(54) METHODS OF INHIBITING AA9 LYTIC POLYSACCHARIDE MONOOXYGENASE CATALYZED INACTIVATION OF ENZYME COMPOSITIONS

(71) Applicant: Novozymes A/S, Bagsvaerd (DK)

(72) Inventors: Keith McFarland, Davis, CA (US); Ani Tejirian, Concord, CA (US); Derek Akerhielm, Farfield, CA (US)

(73) Assignee: Novozymes A/S, Bagsvaerd (DK)

(21) Appl. No.: 15/746,453

(22) PCT Filed: Sep. 2, 2016
(86) PCT No.: PCT/US2016/050075

§ 371 Date: Jan. 22, 2018

Related U.S. Application Data
(60) Provisional application No. 62/214,373, filed on Sep. 4, 2015.

(51) Int. Cl. C13K 1/02; C12N 9/02; C12N 9/08; C12P 19/02; C12P 19/14

(52) U.S. Cl. CPC C13K 1/02; C12N 9/0061; C12N 9/0065; C12P 19/02; C12Y 111/01006; C12P 2201/00; C12P 2203/00; C12Y 110/03002; C12Y 302/01004; C12P 19/14

(57) ABSTRACT

The present invention relates to methods of inhibiting AA9 lytic polysaccharide monooxygenase catalyzed inactivation of an enzyme composition or a component thereof, methods for increasing production of an enzyme composition, and methods for stabilizing an enzyme composition.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to methods of inhibiting AA9 lytic polysaccharide monooxygenase catalyzed inactivation of an enzyme composition or a component thereof, methods for increasing production of an enzyme composition, and methods for stabilizing an enzyme composition.

### Description of the Related Art

Lignocellulosic material provides an attractive platform for generating alternative energy sources to fossil fuels. The conversion of the lignocellulosic material (e.g., from lignocellulosic feedstock) into biofuels has the advantages of the ready availability of large amounts of feedstock, the desirability of avoiding burning or land filling the materials, and the cleanliness of the biofuels (such as ethanol). Wood, agricultural residues, herbaceous crops, and municipal solid wastes have been considered as feedstocks for biofuel production. Once the lignocellulosic material is saccharified and converted to fermentable sugars, e.g., glucose, the fermentable sugars may be fermented by yeast into biofuel, such as ethanol.

New and improved enzymes and enzyme compositions have been developed over the past decade and made saccharification of pretreated cellulosic material more efficient. However, there is a need in the art for further improving the enzyme compositions.

The present invention provides methods of inhibiting AA9 lytic polysaccharide monooxygenase catalyzed inactivation of an enzyme composition or a component thereof, methods for increasing production of an enzyme composition, and methods for stabilizing an enzyme composition.

### SUMMARY OF THE INVENTION

The present invention relates to methods of inhibiting AA9 lytic polysaccharide monooxygenase catalyzed inactivation of an enzyme composition or a component thereof, said method comprising: adding one or more oxidoreductases selected from the group consisting of a catalase, a laccase, a peroxidase, and a superoxide dismutase to the enzyme composition comprising an AA9 lytic polysaccharide monooxygenase and one or more enzyme components, wherein the one or more added oxidoreductases inhibit AA9 lytic polysaccharide monooxygenase catalyzed inactivation of the one or more enzyme components of the enzyme composition.

The present invention also relates to methods for increasing production of an enzyme composition, said methods comprising: (a) fermenting a host cell to produce the enzyme composition in the presence of one or more added oxidoreductases selected from the group consisting of a catalase, a laccases, a peroxidase, and a superoxide dismutase, wherein the enzyme composition comprises an AA9 lytic polysaccharide monooxygenase and one or more enzyme components, wherein the one or more added oxidoreductases inhibit the AA9 lytic polysaccharide monooxygenase catalyzed inactivation of the one or more enzyme components of the enzyme composition, and wherein the amount of the enzyme composition produced in the presence of the one or more added oxidoreductases is higher compared to the amount of the enzyme composition produced in the absence of the added one or more oxidoreductases; and optionally (b) recovering the enzyme composition.

The present invention also relates to methods for stabilizing an enzyme composition, comprising adding one or more oxidoreductases selected from the group consisting of a catalase, a laccases, a peroxidase, and a superoxide dismutase to the enzyme composition, wherein the enzyme composition comprises an AA9 lytic polysaccharide monooxygenase and one or more enzyme components, and wherein the one or more added oxidoreductases inhibit AA9 lytic polysaccharide monooxygenase catalyzed inactivation of the one or more enzyme components of the enzyme composition.

### BRIEF DESCRIPTION OF THE FIGURES

FIG. 1 shows the results of pretreated corn cobs and stover (PCCS) hydrolysis assays (20 g) at 50° C. and pH 5.0 for 5 days with the pH 4.5 fermentation broth filtrates 1, 3, 5, and 7 (Example 1) and the pH 3.5 fermentation broth filtrates 2, 4, 6, and 8 (Example 2).

FIG. 2 shows the results of a fluorescence cellulose decay (FCD) assay of mixtures 1, 3, 5 and 7 (pH 4.5 fermentation, Example 1) after 6 days incubation at 50° C. and pH 5.0.

FIG. 3 shows the results of a FCD assay of mixtures 2, 4, 6 and 8 (pH 3.5 fermentation, Example 2) after 6 days incubation at pH 5.0 and 50° C.

FIG. 4A shows the results of a FCD assay on mixtures 1, 3, 5, and 7 after 4 weeks aseptic storage at 4, 25, 40 and 50° C. and FIG. 4B show the results of a FCD assay on mixtures 2, 4, 6, and 8 after 4 weeks aseptic storage at 4, 25, 40 and 50° C.

FIG. 5 shows the effect of catalase addition during fermentation (mixtures 11 and 12) and no catalase addition during fermentation (mixtures 9 and 10) on performance after 4 week storage at 4, 25, and 40° C. measured by FCD assay at pH 5.0 and 55° C. for 5 days.

FIG. 6 shows the effect of addition of Terminox® Supreme catalase after fermentation on mixture 13 by enzyme replacement at 0%, 0.1%, 0.5%, 1% and 2% w/w catalase protein measured by FCD assay at pH 5.0 and 55° C. for 5 days.

FIG. 7 shows Western blot analysis of filtered fermentation broths 1-8 (lanes 1-8). Lanes 11-16 represent BCA Microplate assay protein-normalized (1 μg) loadings of daily samples from days 2 to 7, respectively, for fermentation 1 (0% catalase over-expression seed B), while lanes 17-22 represent the equivalent samples for fermentation 5 (10% catalase over-expression seed B).

FIG. 8 shows Western blot analysis of filtered fermentation broths 9 (lane 1), 10 (lane 2), 11 (lane 3), and 12 (lanes 4). The un-numbered lane is molecular weight standards in kilodaltons.

### DEFINITIONS

Acetylxylan esterase: The term “acetylxylan esterase” means a carboxylesterase (EC 3.1.1.72) that catalyzes the hydrolysis of acetyl groups from polymeric xylan, acetylated xylose, acetylated glucose, alpha-napthyl acetate, and p-nitrophenyl acetate. Acetylxylan esterase activity can be determined using 0.5 mM p-nitrophenylacetate as substrate in 50 mM sodium acetate pH 5.0 containing 0.01% TWEEN™ 20 (polyoxyethylene sorbitan monolaurate). One unit of acetylxylan esterase is defined as the amount of enzyme capable of releasing 1 μmole of p-nitrophenolate anion per minute at pH 5, 25° C.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Alpha-L-arabinofuranosidase: The term “alpha-L-arabinofuranosidase” means an alpha-L-arabinofuranoside arabinofuranohydrolase (EC 3.2.1.55) that catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,5)-linkages, arabinoxylans, and arabinogalactans. Alpha-L-arabinofuranosidase is also known as arabinosidase, alpha-arabinosidase, alpha-L-arabinosidase, alpha-arabinofuranosidase, polysaccharide alpha-L-arabinofuranosidase, alpha-L-arabinofuranoside hydrolase, L-arabinosidase, or alpha-L-arabinanase. Alpha-L-arabinofuranosidase activity can be determined using 5 mg of medium viscosity wheat arabinoxylan (Megazyme International Ireland, Ltd.) per ml of 100 mM sodium acetate pH 5 in a total volume of 200 μl for 30 minutes at 40° C. followed by arabinose analysis by AMINEX® HPX-87H column chromatography (Bio-Rad Laboratories, Inc.).

Alpha-glucuronidase: The term “alpha-glucuronidase” means an alpha-D-glucosiduronate glucuronohydrolase (EC 3.2.1.139) that catalyzes the hydrolysis of an alpha-D-glucuronoside to D-glucuronate and an alcohol. Alpha-glucuronidase activity can be determined according to de Vries, 1998, J. Bacteriol. 180: 243-249. One unit of alpha-glucuronidase equals the amount of enzyme capable of releasing 1 μmole of glucuronic or 4-O-methylglucuronic acid per minute at pH 5, 40° C.

Auxiliary Activity 9 polypeptide: The term “Auxiliary Activity 9 polypeptide” or “AA9 polypeptide” means a polypeptide classified as a lytic polysaccharide monooxygenase (Quinlan et al., 2011, Proc. Natl. Acad. Sci. USA 108: 15079-15084; Phillips et al., 2011, ACS Chem. Biol. 6: 1399-1406; Li et al., 2012, Structure 20: 1051-1061). AA9 polypeptides were formerly classified into the glycoside hydrolase Family 61 (GH61) according to Henrissat, 1991, Biochem. J. 280: 309-316, and Henrissat and Bairoch, 1996, Biochem. J. 316: 695-696. Such polypeptides are referred to as “AA9 lytic polysaccharide monooxygenases” herein.

AA9 lytic polysaccharide monooxygenases enhance the hydrolysis of a cellulosic material by enzymes having cellulolytic activity. Cellulolytic enhancing activity can be determined by measuring the increase in reducing sugars or the increase of the total of cellobiose and glucose from the hydrolysis of a cellulosic material by cellulolytic enzyme under the following conditions: 1-50 mg of total protein/g of cellulose in pretreated corn stover (PCS), wherein total protein is comprised of 50-99.5% w/w cellulolytic enzyme protein and 0.5-50% w/w protein of an AA9 polypeptide for 1-7 days at a suitable temperature, such as 40° C.-80° C., e.g., 40° C., 45° C., 50° C., 55° C., 60° C., 65° C., 70° C., 75° C., or 80° C. and a suitable pH, such as 4-9, e.g., 4.5, 5.0, 5.5, 6.0, 6.5, 7.0, 7.5, 8.0, 8.5, or 9.0, compared to a control hydrolysis with equal total protein loading without cellulolytic enhancing activity (1-50 mg of cellulolytic protein/g of cellulose in PCS).

Cellulolytic enhancing activity can be determined using a mixture of CELLUCLAST™ 1.5L (Novozymes A/S, Bagsværd, Denmark) and beta-glucosidase as the source of the cellulolytic activity, wherein the beta-glucosidase is present at a weight of at least 2-5% protein of the cellulase protein loading. In one aspect, the beta-glucosidase is an Aspergillus oryzae beta-glucosidase (e.g., recombinantly produced in Aspergillus oryzae according to WO 02/095014). In another aspect, the beta-glucosidase is an Aspergillus fumigatus beta-glucosidase (e.g., recombinantly produced in Aspergillus oryzae as described in WO 02/095014).

Cellulolytic enhancing activity can also be determined by incubating an AA9 polypeptide with 0.5% phosphoric acid swollen cellulose (PASC), 100 mM sodium acetate pH 5, 1 mM MnSO₄, 0.1% gallic acid, 0.025 mg/ml of Aspergillus fumigatus beta-glucosidase, and 0.01% TRITON® X-100 (4-(1,1,3,3-tetramethylbutyl)phenyl-polyethylene glycol) for 24-96 hours at 40° C. followed by determination of the glucose released from the PASC.

Cellulolytic enhancing activity can also be determined according to WO 2013/028928 for high temperature compositions.

AA9 lytic polysaccharide monooxygenases enhance the hydrolysis of a cellulosic material catalyzed by enzymes having cellulolytic activity by reducing the amount of cellulolytic enzyme required to reach the same degree of hydrolysis preferably at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold.

AA9 lytic polysaccharide monooxygenases can be used in the presence of a soluble activating divalent metal cation according to WO 2008/151043 or WO 2012/122518, e.g., manganese or copper.

AA9 lytic polysaccharide monooxygenases can also be used in the presence of a dioxy compound, a bicylic compound, a heterocyclic compound, a nitrogen-containing compound, a quinone compound, a sulfur-containing compound, or a liquor obtained from a pretreated cellulosic or hemicellulosic material such as pretreated corn stover (WO 2012/021394, WO 2012/021395, WO 2012/021396, WO 2012/021399, WO 2012/021400, WO 2012/021401, WO 2012/021408, and WO 2012/021410).

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. Beta-glucosidase activity can be determined using p-nitrophenyl-beta-D-glucopyranoside as substrate according to the procedure of Venturi et al., 2002, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20.

Beta-xylosidase: The term “beta-xylosidase” means a beta-D-xyloside xylohydrolase (E.C. 3.2.1.37) that catalyzes the exo-hydrolysis of short beta (1→4)-xylooligosaccharides to remove successive D-xylose residues from non-reducing termini. Beta-xylosidase activity can be determined using 1 mM p-nitrophenyl-beta-D-xyloside as substrate in 100 mM sodium citrate containing 0.01% TWEEN® 20 at pH 5, 40° C. One unit of beta-xylosidase is defined as 1.0 μmole of p-nitrophenolate anion produced per minute at 40° C., pH 5 from 1 mM p-nitrophenyl-beta-D-xyloside in 100 mM sodium citrate containing 0.01% TWEEN® 20.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Catalase: The term “catalase” means a hydrogen-peroxide:hydrogen-peroxide oxidoreductase (E.C. 1.11.1.6 or E.C. 1.11.1.21) that catalyzes the conversion of two hydrogen peroxides to oxygen and two waters.

Catalase activity can be determined by monitoring the degradation of hydrogen peroxide at 240 nm based on the following reaction:

2H₂O₂→2H₂O+O₂

The reaction is conducted in 50 mM phosphate pH 7 at 25° C. with 10.3 mM substrate (H₂O₂). Absorbance is monitored spectrophotometrically within 16-24 seconds, which should correspond to an absorbance reduction from 0.45 to 0.4. One catalase activity unit can be expressed as one μmole of H₂O₂ degraded per minute at pH 7.0 and 25° C.

Cellobiohydrolase: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing end (cellobiohydrolase I) or non-reducing end (cellobiohydrolase II) of the chain (Teeri, 1997, Trends in Biotechnology 15: 160-167; Teeri et al., 1998, Biochem. Soc. Trans. 26: 173-178). Cellobiohydrolase activity can be determined according to the procedures described by Lever et al., 1972, Anal. Biochem. 47: 273-279; van Tilbeurgh et al., 1982, FEBS Letters 149: 152-156; van Tilbeurgh and Claeyssens, 1985, FEBS Letters 187: 283-288; and Tomme et al., 1988, Eur. J. Biochem. 170: 575-581.

Cellulolytic enzyme or cellulase: The term “cellulolytic enzyme” or “cellulase” means one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24: 452-481. Total cellulolytic enzyme activity can be measured using insoluble substrates, including Whatman N [symbol] 1 filter paper, microcrystalline cellulose, bacterial cellulose, algal cellulose, cotton, pretreated lignocellulose, etc. The most common total cellulolytic activity assay is the filter paper assay using Whatman N [symbol] 1 filter paper as the substrate. The assay was established by the International Union of Pure and Applied Chemistry (IUPAC) (Ghose, 1987, Pure Appl. Chem. 59: 257-68).

Cellulolytic enzyme activity can be determined by measuring the increase in production/release of sugars during hydrolysis of a cellulosic material by cellulolytic enzyme(s) under the following conditions: 1-50 mg of cellulolytic enzyme protein/g of cellulose in pretreated corn stover (PCS) (or other pretreated cellulosic material) for 3-7 days at a suitable temperature such as 40° C.-80° C., e.g., 40° C., 45° C., 50° C., 55° C., 60° C., 65° C., 70° C., 75° C., or 80° C., and a suitable pH, such as 4-9, e.g., 4.0, 4.5, 5.0, 5.5, 6.0, 6.5, 7.0, 7.5, 8.0, 8.5, or 9.0, compared to a control hydrolysis without addition of cellulolytic enzyme protein. Typical conditions are 1 ml reactions, washed or unwashed PCS, 5% insoluble solids (dry weight), 50 mM sodium acetate pH 5, 1 mM MnSO₄, 50° C., 55° C., or 60° C., 72 hours, sugar analysis by AMINEX® HPX-87H column chromatography (Bio-Rad Laboratories, Inc.).

Cellulosic material: The term “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cellulose is generally found, for example, in the stems, leaves, hulls, husks, and cobs of plants or leaves, branches, and wood of trees. The cellulosic material can be, but is not limited to, agricultural residue, herbaceous material (including energy crops), municipal solid waste, pulp and paper mill residue, waste paper, and wood (including forestry residue) (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, N.Y.). It is understood herein that the cellulose may be in the form of lignocellulose, a plant cell wall material containing lignin, cellulose, and hemicellulose in a mixed matrix. In one aspect, the cellulosic material is any biomass material. In another aspect, the cellulosic material is lignocellulose (lignocellulosic material), which comprises cellulose, hemicelluloses, and lignin.

In an embodiment, the cellulosic material is agricultural residue, herbaceous material (including energy crops), municipal solid waste, pulp and paper mill residue, waste paper, or wood (including forestry residue).

In another embodiment, the cellulosic material is arundo, bagasse, bamboo, corn cob, corn fiber, corn stover, miscanthus, rice straw, sugar cane straw, switchgrass, or wheat straw.

In another embodiment, the cellulosic material is aspen, eucalyptus, fir, pine, poplar, spruce, or willow.

In another embodiment, the cellulosic material is algal cellulose, bacterial cellulose, cotton linter, filter paper, microcrystalline cellulose (e.g., AVICEL®), or phosphoric-acid treated cellulose.

In another embodiment, the cellulosic material is an aquatic biomass. As used herein the term “aquatic biomass” means biomass produced in an aquatic environment by a photosynthesis process. The aquatic biomass can be algae, emergent plants, floating-leaf plants, or submerged plants.

The cellulosic material may be used as is or may be subjected to pretreatment, using conventional methods known in the art. In a preferred aspect, the cellulosic material is pretreated.

Endoglucanase: The term “endoglucanase” means a 4-(1,3;1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3-1,4 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). Endoglucanase activity can also be determined using carboxymethyl cellulose (CMC) as substrate according to the procedure of Ghose, 1987, Pure and Appl. Chem. 59: 257-268, at pH 5, 40° C.

Feruloyl esterase: The term “feruloyl esterase” means a 4-hydroxy-3-methoxycinnamoyl-sugar hydrolase (EC 3.1.1.73) that catalyzes the hydrolysis of 4-hydroxy-3-methoxycinnamoyl (feruloyl) groups from esterified sugar, which is usually arabinose in natural biomass substrates, to produce ferulate (4-hydroxy-3-methoxycinnamate). Feruloyl esterase (FAE) is also known as ferulic acid esterase, hydroxycinnamoyl esterase, FAE-III, cinnamoyl ester hydrolase, FAEA, cinnAE, FAE-I, or FAE-II. Feruloyl esterase activity can be determined using 0.5 mM p-nitrophenylferulate as substrate in 50 mM sodium acetate pH 5.0. One unit of feruloyl esterase equals the amount of enzyme capable of releasing 1 μmole of p-nitrophenolate anion per minute at pH 5, 25° C.

Fragment: The term “fragment” means a polypeptide having one or more (e.g., several) amino acids absent from the amino and/or carboxyl terminus of the mature polypeptide thereof, wherein the fragment has cellulolytic enhancing activity. In one aspect, a fragment contains at least 85% of the amino acid residues, e.g., at least 90% of the amino acid residues or at least 95% of the amino acid residues of the mature polypeptide of an AA9 lytic polysaccharide monooxygenase.

Hemicellulolytic enzyme or hemicellulase: The term “hemicellulolytic enzyme” or “hemicellulase” means one or more (e.g., several) enzymes that hydrolyze a hemicellulosic material. See, for example, Shallom and Shoham, 2003, Current Opinion In Microbiology 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates for these enzymes, hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752, at a suitable temperature such as 40° C.-80° C., e.g., 40° C., 45° C., 50° C., 55° C., 60° C., 65° C., 70° C., 75° C., or 80° C., and a suitable pH such as 4-9, e.g., 4.0, 4.5, 5.0, 5.5, 6.0, 6.5, 7.0, 7.5, 8.0, 8.5, or 9.0.

Hemicellulosic material: The term “hemicellulosic material” means any material comprising hemicelluloses. Hemicelluloses include xylan, glucuronoxylan, arabinoxylan, glucomannan, and xyloglucan. These polysaccharides contain many different sugar monomers. Sugar monomers in hemicellulose can include xylose, mannose, galactose, rhamnose, and arabinose. Hemicelluloses contain most of the D-pentose sugars. Xylose is in most cases the sugar monomer present in the largest amount, although in softwoods mannose can be the most abundant sugar. Xylan contains a backbone of beta-(1-4)-linked xylose residues. Xylans of terrestrial plants are heteropolymers possessing a beta-(1-4)-D-xylopyranose backbone, which is branched by short carbohydrate chains. They comprise D-glucuronic acid or its 4-O-methyl ether, L-arabinose, and/or various oligosaccharides, composed of D-xylose, L-arabinose, D- or L-galactose, and D-glucose. Xylan-type polysaccharides can be divided into homoxylans and heteroxylans, which include glucuronoxylans, (arabino)glucuronoxylans, (glucurono)arabinoxylans, arabinoxylans, and complex heteroxylans. See, for example, Ebringerova et al., 2005, Adv. Polym. Sci. 186: 1-67. Hemicellulosic material is also known herein as “xylan-containing material”.

Sources for hemicellulosic material are essentially the same as those for cellulosic material described herein.

In a preferred aspect, the hemicellulosic material is lignocellulose (lignocellulosic material).

Laccase: The term “laccase” means a benzenediol:oxygen oxidoreductase (E.C. 1.10.3.2) that catalyzes the following reaction: 1,2- or 1,4-benzenediol+O₂=1,2- or 1,4-benzosemiquinone+2H₂O.

Laccase activity can be determined by the oxidation of syringaldazine (4,4′-[azinobis(methanylylidene)]bis(2,6-dimethoxyphenol)) to the corresponding quinone 4,4′-[azobis(methanylylidene])bis(2,6-dimethoxycyclohexa-2,5-dien-1-one) by laccase. The reaction (shown below) is detected by an increase in absorbance at 530 nm.

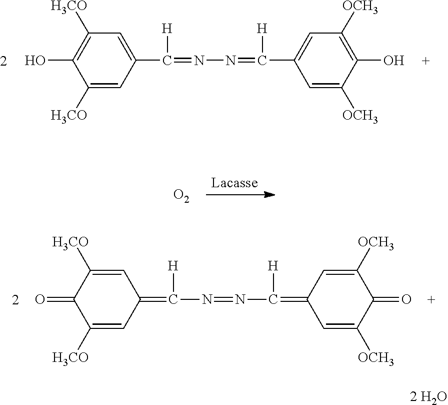 [SMILES: COc1cc(C=NN=Cc2cc(OC)c(O)c(OC)c2)cc(OC)c1O ; "O2" ; COC1=CC(=CN=NC=C2C=C(OC)C(=O)C(OC)=C2)C=C(OC)C1=O ; "2 H2O"]

The reaction is conducted in 23 mM MES pH 5.5 at 30° C. with 19 μM substrate (syringaldazine) and 1 g/L polyethylene glycol (PEG) 6000. The sample is placed in a spectrophotometer and the change in absorbance is measured at 530 nm every 15 seconds up to 90 seconds. One laccase unit is the amount of enzyme that catalyzes the conversion of 1 μmole syringaldazine per minute under the specified analytical conditions.

Mature polypeptide: The term “mature polypeptide” means a polypeptide in its final form following translation and any post-translational modifications, such as N-terminal processing, C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide.

Mature polypeptide coding sequence: The term “mature polypeptide coding sequence” means a polynucleotide that encodes a mature polypeptide having enzyme or biological activity. The term “mature polypeptide coding sequence” herein shall be understood to include the cDNA sequence of the genomic DNA sequence or the genomic DNA sequence of the cDNA sequence.

Peroxidase: The term “peroxidase” means an enzyme that converts a peroxide, e.g., hydrogen peroxide, to a less oxidative species, e.g., water. It is understood herein that a peroxidase encompasses a peroxide-decomposing enzyme. The term “peroxide-decomposing enzyme” is defined herein as a donor:peroxide oxidoreductase (E.C. number 1.11.1.x, wherein x=1-3, 5, 7-19, or 21) that catalyzes the reaction reduced substrate (2e⁻)+ROOR′→oxidized substrate+ROH+R′OH; such as horseradish peroxidase that catalyzes the reaction phenol+H₂O₂→quinone+H₂O, and catalase that catalyzes the reaction H₂O₂+H₂O₂→O₂+2H₂O. In addition to hydrogen peroxide, other peroxides may also be decomposed by these enzymes.

Peroxidase activity can be determined by measuring the oxidation of 2,2′-azino-bis(3-ethylbenzthiazoline-6-sulfonic acid (ABTS) by a peroxidase in the presence of hydrogen peroxide as shown below. The reaction product ABTSₒₓ forms a blue-green color which can be quantified at 418 nm.

H₂O₂+2ABTS_{red}+2H⁺→2H₂O+2ABTSₒₓ

The reaction is conducted in 0.1 M phosphate pH 7 at 30° C. with 1.67 mM substrate (ABTS), 1.5 g/L TRITON® X-405, 0.88 mM hydrogen peroxide, and approximately 0.040 units enzyme per ml. The sample is placed in a spectrophotometer and the change in absorbance is measured at 418 nm from 15 seconds up to 60 seconds. One peroxidase unit can be expressed as the amount of enzyme required to catalyze the conversion of 1 μmole of hydrogen peroxide per minute under the specified analytical conditions.

Pretreated cellulosic or hemicellulosic material: The term “pretreated cellulosic or hemicellulosic material” means a cellulosic or hemicellulosic material derived from biomass by treatment with heat and dilute sulfuric acid, alkaline pretreatment, neutral pretreatment, or any pretreatment known in the art.

Pretreated corn cobs and stover: The term “pretreated corn cobs and stover” or “PCCS” means a cellulosic material derived from corn cobs and stover by treatment with heat and dilute sulfuric acid, alkaline pretreatment, neutral pretreatment, or any pretreatment known in the art.

Pretreated corn stover: The term “pretreated corn stover” or “PCS” means a cellulosic material derived from corn stover by treatment with heat and dilute sulfuric acid, alkaline pretreatment, neutral pretreatment, or any pretreatment known in the art.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are a gap open penalty of 10, a gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are a gap open penalty of 10, a gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment)

Stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 45° C.

The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 50° C.

The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 55° C.

The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 60° C.

The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 65° C.

The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 70° C.

Subsequence: The term “subsequence” means a polynucleotide having one or more (e.g., several) nucleotides absent from the 5′ and/or 3′ end of a mature polypeptide coding sequence, wherein the subsequence encodes a fragment having cellulolytic enhancing activity. In one aspect, a subsequence contains at least 85% of the nucleotides, e.g., at least 90% of the nucleotides or at least 95% of the nucleotides of the mature polypeptide coding sequence of an AA9 lytic polysaccharide monooxygenase.

Superoxide dismutase: The term “superoxide dismutase” means an enzyme (E.C. 1.15.1.1) that alternately catalyzes the dismutation (or partitioning) of the superoxide (O₂⁻) radical into either ordinary molecular oxygen (O₂) or hydrogen peroxide (H₂O₂) as follows:

Cu²⁺-SOD+O₂⁻→Cu⁺-SOD+O₂

Cu⁺-SOD+O₂⁻+2H⁺→Cu²⁺-SOD+H₂O₂

Superoxide dismutase activity can be determined according to Beauchamp and Fridovich, 1971, Anal. Biochem. 44: 276-287.

Xylan-containing material: The term “xylan-containing material” means any material comprising a plant cell wall polysaccharide containing a backbone of beta-(1-4)-linked xylose residues. Xylans of terrestrial plants are heteropolymers possessing a beta-(1-4)-D-xylopyranose backbone, which is branched by short carbohydrate chains. They comprise D-glucuronic acid or its 4-O-methyl ether, L-arabinose, and/or various oligosaccharides, composed of D-xylose, L-arabinose, D- or L-galactose, and D-glucose. Xylan-type polysaccharides can be divided into homoxylans and heteroxylans, which include glucuronoxylans, (arabino)glucuronoxylans, (glucurono)arabinoxylans, arabinoxylans, and complex heteroxylans. See, for example, Ebringerova et al., 2005, Adv. Polym. Sci. 186: 1-67. In a preferred aspect, the xylan-containing material is lignocellulose.

Xylan degrading activity or xylanolytic activity: The term “xylan degrading activity” or “xylanolytic activity” means a biological activity that hydrolyzes xylan-containing material. The two basic approaches for measuring xylanolytic activity include: (1) measuring the total xylanolytic activity, and (2) measuring the individual xylanolytic activities (e.g., endoxylanases, beta-xylosidases, arabinofuranosidases, alpha-glucuronidases, acetylxylan esterases, feruloyl esterases, and alpha-glucuronyl esterases). Recent progress in assays of xylanolytic enzymes was summarized in several publications including Biely and Puchard, 2006, Journal of the Science of Food and Agriculture 86(11): 1636-1647; Spanikova and Biely, 2006, FEBS Letters 580(19): 4597-4601; Herrimann et al., 1997, Biochemical Journal 321: 375-381.

Total xylan degrading activity can be measured by determining the reducing sugars formed from various types of xylan, including, for example, oat spelt, beechwood, and larchwood xylans, or by photometric determination of dyed xylan fragments released from various covalently dyed xylans. A common total xylanolytic activity assay is based on production of reducing sugars from polymeric 4-O-methyl glucuronoxylan as described in Bailey et al., 1992, Interlaboratory testing of methods for assay of xylanase activity, Journal of Biotechnology 23(3): 257-270. Xylanase activity can also be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

Xylan degrading activity can be determined by measuring the increase in hydrolysis of birchwood xylan (Sigma Chemical Co., Inc.) by xylan-degrading enzyme(s) under the following typical conditions: 1 ml reactions, 5 mg/ml substrate (total solids), 5 mg of xylanolytic protein/g of substrate, 50 mM sodium acetate pH 5, 50° C., 24 hours, sugar analysis using p-hydroxybenzoic acid hydrazide (PHBAH) assay as described by Lever, 1972, Anal. Biochem. 47: 273-279.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. Xylanase activity can be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

Reference to “about” a value or parameter herein includes aspects that are directed to that value or parameter per se. For example, description referring to “about X” includes the aspect “X”.

As used herein and in the appended claims, the singular forms “a,” “or,” and “the” include plural referents unless the context clearly dictates otherwise. It is understood that the aspects of the invention described herein include “consisting” and/or “consisting essentially of” aspects.

Unless defined otherwise or clearly indicated by context, all technical and scientific terms used herein have the same meaning as commonly understood by one of ordinary skill in the art to which this invention belongs.

### DETAILED DESCRIPTION OF THE INVENTION

The present invention relates to methods of inhibiting AA9 lytic polysaccharide monooxygenase catalyzed inactivation of an enzyme composition or a component thereof, said method comprising: adding one or more oxidoreductases selected from the group consisting of a catalase, a laccase, a peroxidase, and a superoxide dismutase to the enzyme composition comprising an AA9 lytic polysaccharide monooxygenase and one or more enzyme components, wherein the one or more added oxidoreductases inhibit AA9 lytic polysaccharide monooxygenase catalyzed inactivation of the one or more enzyme components of the enzyme composition.

The present invention also relates to methods for increasing production of an enzyme composition, said methods comprising: (a) fermenting a host cell to produce the enzyme composition in the presence of one or more added oxidoreductases selected from the group consisting of a catalase, a laccases, a peroxidase, and a superoxide dismutase, wherein the enzyme composition comprises an AA9 lytic polysaccharide monooxygenase and one or more enzyme components, wherein the one or more added oxidoreductases inhibit the AA9 lytic polysaccharide monooxygenase catalyzed inactivation of the one or more enzyme components of the enzyme composition, and wherein the amount of the enzyme composition produced in the presence of the one or more added oxidoreductases is higher compared to the amount of the enzyme composition produced in the absence of the added one or more oxidoreductases; and optionally (b) recovering the enzyme composition. In one aspect, the one or more added oxidoreductases are added to the fermentation. In another aspect, the one or more added oxidoreductases are recombinantly produced by the host cell. In another aspect, the one or more added oxidoreductases are recombinantly produced by co-culture of the recombinant cell with a second host cell. In another aspect, the one or more added oxidoreductases are added to the fermentation and recombinantly produced by the host cell. In another aspect, the one or more added oxidoreductases are added to the fermentation and recombinantly produced by co-culture of the recombinant cell with a second host cell. In another aspect, the one or more added oxidoreductases are recombinantly produced by the host cell and recombinantly produced by co-culture of the recombinant cell with a second host cell. In another aspect, the one or more added oxidoreductases are added to the fermentation, recombinantly produced by the host cell, and recombinantly produced by co-culture of the recombinant cell with a second host cell.

The present invention also relates to methods for stabilizing an enzyme composition, comprising adding one or more oxidoreductases selected from the group consisting of a catalase, a laccases, a peroxidase, and a superoxide dismutase to the enzyme composition, wherein the enzyme composition comprises an AA9 lytic polysaccharide monooxygenase and one or more enzyme components, and wherein the one or more added oxidoreductases inhibit AA9 lytic polysaccharide monooxygenase catalyzed inactivation of the one or more enzyme components of the enzyme composition.

The present invention allows for the production of AA9 lytic polysaccharide monooxygenases in high amounts, while inhibiting AA9 lytic polysaccharide monooxygenase catalyzed inactivation of components of an enzyme composition. Without being bound by any theory, catalase, for example, converts hydrogen peroxide produced by the AA9 enzyme to water and oxygen, blocking the formation of reactive oxygen species that can modify proteins, including the enzyme components of the enzyme composition. The proteins modified by the reactive oxygen species may then be destabilized or inactivated. The modified proteins may also be degraded by proteases that may be present in the enzyme composition. The inhibition of AA9 lytic polysaccharide monooxygenase catalyzed inactivation of components of an enzyme composition results in higher quality enzyme compositions at the end of fermentation and recovery. Since inhibition with catalase is possible at higher pH, e.g., pH 4.5, fermentations can be performed under conditions that produce more protein than at lower pH. Moreover, inhibition with catalase insures more stable enzyme compositions, as the un-modified enzymes are more likely stable to proteases that may be present in the enzyme composition.

In one aspect, the inhibition of the AA9 lytic polysaccharide monooxygenase catalyzed inactivation is higher in the presence of the one or more added oxidoreductases compared to the absence of the one or more added oxidoreductases. In one aspect, the oxidoreductase, e.g., catalase, laccase, peroxidase, and superoxide dismutase, inhibits AA9 lytic polysaccharide monooxygenase catalyzed inactivation of an enzyme composition or a component thereof at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The inhibition of the AA9 lytic polysaccharide monooxygenase catalyzed inactivation of components of an enzyme composition can result in higher yields of fermentable sugars, e.g., glucose, from saccharification of a cellulosic material. Saccharification can be performed according to WO 2013/028928. In one aspect, the yield of fermentable sugar, e.g., glucose, is increased at least 1%, at least 2%, at least 3%, at least 4%, at least 5%, at least 10%, at least 15%, or at least 20%.

In another aspect, the presence of oxidoreductase, e.g., catalase, laccase, peroxidase, and superoxide dismutase, increases production of an active enzyme composition or an active component thereof at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

In another aspect, an enzyme composition stabilized with one or more oxidoreductases has a higher stability (retention of enzyme activity) at 25° C. for 4 weeks of at least 1%, at least 2%, at least 3%, at least 5%, at least 7%, at least 9%, at least 10%, at least 15%, at least 20%, at least 40%, at least 60%, at least 80%, or at least 100% compared to an enzyme composition not containing the one or more oxidoreductases. In another aspect, an enzyme composition stabilized with one or more oxidoreductases has a higher stability at 40° C. for 4 weeks of at least 1%, at least 2%, at least 3%, at least 5%, at least 7%, at least 9%, at least 10%, at least 12%, at least 15%, at least 20%, at least 40%, at least 60%, at least 80%, or at least 100% compared to an enzyme composition not containing the one or more oxidoreductases. In another aspect, an enzyme composition stabilized with one or more oxidoreductases has a higher stability at 50° C. for 4 weeks of at least 1%, at least 2%, at least 3%, at least 5%, at least 7%, at least 9%, at least 10%, at least 15%, at least 20%, at least 40%, at least 60%, at least 80%, or at least 100% compared to an enzyme composition not containing the one or more oxidoreductases.

### AA9 Lytic Polysaccharide Monooxygenases

The AA9 lytic polysaccharide monooxygenase may be any AA9 lytic polysaccharide monooxygenase. The AA9 lytic polysaccharide monooxygenase may be native or foreign to the strain from which the enzyme composition is derived or isolated, such as a strain of Aspergillus niger, Aspergillus oryzae, Chrysosporium lucknowense (Myceliophthora thermophila), Fusarium venenatum, Humicola insolens, Talaromyces emersonii, or Trichoderma reesei. In an embodiment, the AA9 lytic polysaccharide monooxygenase is a recombinant AA9 polypeptide. In another embodiment, the AA9 lytic polysaccharide monooxygenase is not of the same origin as the enzyme composition's host cell, e.g., not of Trichoderma origin, such as not of Trichoderma reesei origin. In an embodiment, the AA9 lytic polysaccharide monooxygenase is produced recombinantly as part of the enzyme composition, e.g., produced by the Trichoderma reesei host cell producing the enzyme composition.

Examples of AA9 lytic polysaccharide monooxygenases include, but are not limited to, AA9 lytic polysaccharide monooxygenases from Acrophialophora fusispora (WO 2013/043910), Aspergillus aculeatus (WO 2012/030799), Aspergillus fumigatus (WO 2010/138754), Aurantiporus alborubescens (WO 2012/122477), Chaetomium thermophilum (WO 2012/101206), Corynascus sepedonium (WO 2013/043910), Humicola insolens (WO 2012/146171), Malbranchea cinnamomea (WO 2012/101206), Myceliophthora thermophila (WO 2009/085935, WO 2009/085859, WO 2009/085864, WO 2009/085868, and WO 2009/033071), Penicillium pinophilum (WO 2011/005867), Penicillium sp. (WO 2011/041397 and WO 2012/000892), Penicillium thomii (WO 2012/122477), Talaromyces emersonii (WO 2012/000892), Talaromyces leycettanus (WO 2012/101206), Talaromyces stipitatus (WO 2012/135659), Talaromyces thermophilus (WO 2012/129697 and WO 2012/130950), Thermoascus aurantiacus (WO 2005/074656 and WO 2010/065830), Thermoascus crustaceous (WO 2011/041504), Thermoascus sp. (WO 2011/039319), Thermomyces lanuginosus (WO 2012/113340, WO 2012/129699, WO 2012/130964, and WO 2012/129699), Thielavia terrestris (WO 2005/074647, WO 2008/148131, and WO 2011/035027), Trichoderma reesei (WO 2007/089290 and WO 2012/149344), and Trichophaea saccata (WO 2012/122477).

Non-limiting examples of AA9 lytic polysaccharide monooxygenases are AA9 lytic polysaccharide monooxygenases from Acrophialophora fusispora (GeneSeqP: BAM80382); Aspergillus aculeatus (GeneSeqP: AZT94039, GeneSeqP: AZT94041, GeneSeqP: AZT94043, GeneSeqP: AZT94045, GeneSeqP: AZT94047, GeneSeqP: AZT94049, GeneSeqP: AZT94051); Aspergillus fumigatus (GeneSeqP: AYM96878); Aspergillus niveus (GeneSeqP: BBE80792); Aurantiporus alborubescens (GeneSeqP: AZZ98498, GeneSeqP: AZZ98500); Chaetomium thermophilum (GeneSeqP: AZY42252); Corynascus sepedonium (GeneSeqP: BAM80384, GeneSeqP: BAM80386); Humicola insolens (GeneSeqP: BAE45292, GeneSeqP: BAE45294, GeneSeqP: BAE45296, GeneSeqP: BAE45298, GeneSeqP: BAE45300, GeneSeqP: BAE45302, GeneSeqP: BAE45304, GeneSeqP: BAE45306, GeneSeqP: BAE45308, GeneSeqP: BAE45310, GeneSeqP: BAE45312, GeneSeqP: BAE45314, GeneSeqP: BAE45316, GeneSeqP: BAE45318, GeneSeqP: BAE45320, GeneSeqP: BAE45322, GeneSeqP: BAE45324, GeneSeqP: BAE45326, GeneSeqP: BAE45328, GeneSeqP: BAE45330, GeneSeqP: BAE45332, GeneSeqP: BAE45334, GeneSeqP: BAE45336, GeneSeqP: BAE45338, GeneSeqP: BAE45340, GeneSeqP: BAE45342, GeneSeqP: BAE45344); Malbranchea cinnamomea (GeneSeqP: AZY42250); Myceliophthora thermophila (GeneSeqP: AXD75715, GeneSeqP: AXD75717, GeneSeqP: AXD58945, GeneSeqP: AXD80944, GeneSeqP: AXF00393); Penicillium sp. (GeneSeqP: AZG65226); Penicillium emersonii (GeneSeqP: BAM92736); Malbranchea cinnamomea (GeneSeqP: BAO18037, GeneSeqP: BAO18039, GeneSeqP: BAO18041, GeneSeqP: BAO18043, GeneSeqP: BAO18045, GeneSeqP: BAO18047, GeneSeqP: BAO18049, GeneSeqP: BAO18051, GeneSeqP: BAO18053); Myceliophthora fergusii (GeneSeqP: BAO17567, GeneSeqP: BAO17569, GeneSeqP: BAO17571, GeneSeqP: BAO17573, GeneSeqP: BAO17575, GeneSeqP: BAO17577, GeneSeqP: BAO17579, GeneSeqP: BAO17581, GeneSeqP: BAO17583, GeneSeqP: BAO17585, GeneSeqP: BAO17587, GeneSeqP: BAO17589, GeneSeqP: BAO17591, GeneSeqP: BAO17593, GeneSeqP: BAO17595, GeneSeqP: BAO17597); Penicillium pinophilum (GeneSeqP: AYN30445); Penicillium thomii (GeneSeqP: AZZ98506); Talaromyces emersonii (GeneSeqP: AZR89286); Talaromyces leycettanus (GeneSeqP: AZY42258); Talaromyces stipitatus (GeneSeqP: BAD71945); Talaromyces thermophilus (GeneSeqP: BAA95296, GeneSeqP: BAA22810); Thermoascus crustaceus (GeneSeqP: AZG67666, GeneSeqP: AZG67668, GeneSeqP: AZG67670); Thermoascus sp. (GeneSeqP: AZG48808); Thermoascus aurantiacus (GeneSeqP: AZJ19467, GeneSeqP: AYD12322); Trichoderma reesei (GeneSeqP: AFY26868, GeneSeqP: BAF28697); Thermomyces lanuginosus (GeneSeqP: AZZ14902, GeneSeqP: AZZ14904, GeneSeqP: AZZ14906); Thielavia terrestris (GeneSeqP: AEB90517, GeneSeqP: AEB90519, GeneSeqP: AEB90521, GeneSeqP: AEB90523, GeneSeqP: AEB90525, GeneSeqP: AUM21652, GeneSeqP: AZG26658, GeneSeqP: AZG26660, GeneSeqP: AZG26662, GeneSeqP: AZG26664, GeneSeqP: AZG26666, GeneSeqP: AZG26668, GeneSeqP: AZG26670, GeneSeqP: AZG26672, GeneSeqP: AZG26674, GeneSeqP: AZG26676, GeneSeqP: AZG26678); and Trichophaea saccata (GeneSeqP: AZZ98502, GeneSeqP: AZZ98504). The accession numbers are incorporated herein in their entirety.

In one aspect, the AA9 lytic polysaccharide monooxygenase has a sequence identity to the mature polypeptide of an AA9 lytic polysaccharide monooxygenase disclosed herein of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, which have AA9 lytic polysaccharide monooxygenase activity.

In another aspect, the amino acid sequence of the AA9 lytic polysaccharide monooxygenase differs by up to 10 amino acids, e.g., 1, 2, 3, 4, 5, 6, 7, 8, 9, or 10 from the mature polypeptide of an AA9 lytic polysaccharide monooxygenase disclosed herein.

In another aspect, the AA9 lytic polysaccharide monooxygenase comprises or consists of the amino acid sequence of an AA9 lytic polysaccharide monooxygenase disclosed herein.

In another aspect, the AA9 lytic polysaccharide monooxygenase comprises or consists of the mature polypeptide of an AA9 lytic polysaccharide monooxygenase disclosed herein.

In another embodiment, the AA9 lytic polysaccharide monooxygenase is an allelic variant of a AA9 lytic polysaccharide monooxygenase disclosed herein.

In another aspect, the AA9 lytic polysaccharide monooxygenase is a fragment containing at least 85% of the amino acid residues, e.g., at least 90% of the amino acid residues or at least 95% of the amino acid residues of the mature polypeptide of a AA9 lytic polysaccharide monooxygenase disclosed herein.

In another aspect, the AA9 lytic polysaccharide monooxygenase is encoded by a polynucleotide that hybridizes under very low, low, medium, medium-high, high, or very high stringency conditions with the mature polypeptide coding sequence or the full-length complement thereof of an AA9 lytic polysaccharide monooxygenase disclosed herein (Sambrook et al., 1989, supra).

The polynucleotide encoding a AA9 lytic polysaccharide monooxygenase, or a subsequence thereof, as well as the polypeptide of a AA9 lytic polysaccharide monooxygenase, or a fragment thereof, may be used to design nucleic acid probes to identify and clone DNA encoding a AA9 lytic polysaccharide monooxygenase from strains of different genera or species according to methods well known in the art. In particular, such probes can be used for hybridization with the genomic DNA or cDNA of a cell of interest, as described supra.

For purposes of the present invention, hybridization indicates that the polynucleotide hybridizes to a labeled nucleic acid probe under very low to very high stringency conditions. Molecules to which the nucleic acid probe hybridizes under these conditions can be detected using, for example, X-ray film or any other detection means known in the art.

In one aspect, the nucleic acid probe is the mature polypeptide coding sequence of a AA9 lytic polysaccharide monooxygenase.

In another aspect, the nucleic acid probe is a polynucleotide that encodes a full-length AA9 lytic polysaccharide monooxygenase; the mature polypeptide thereof; or a fragment thereof.

In another aspect, the AA9 lytic polysaccharide monooxygenase is encoded by a polynucleotide having a sequence identity to the mature polypeptide coding sequence of an AA9 lytic polysaccharide monooxygenase disclosed herein of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

The AA9 lytic polysaccharide monooxygenase may be a hybrid polypeptide in which a region of one polypeptide is fused at the N-terminus or the C-terminus of a region of another polypeptide or a fusion polypeptide or cleavable fusion polypeptide in which another polypeptide is fused at the N-terminus or the C-terminus of the AA9 lytic polysaccharide monooxygenase, as described herein.

The AA9 lytic polysaccharide monooxygenase may be obtained from microorganisms of any genus. For purposes of the present invention, the term “obtained from” as used herein in connection with a given source shall mean that the AA9 lytic polysaccharide monooxygenase encoded by a polynucleotide is produced by the source or by a strain in which the polynucleotide from the source has been inserted. In one embodiment, the AA9 lytic polysaccharide monooxygenase is secreted extracellularly.

The AA9 lytic polysaccharide monooxygenase may be a bacterial AA9 lytic polysaccharide monooxygenase. For example, the AA9 lytic polysaccharide monooxygenase may be a Gram-positive bacterial polypeptide such as a Bacillus, Clostridium, Enterococcus, Geobacillus, Lactobacillus, Lactococcus, Oceanobacillus, Staphylococcus, Streptococcus, or Streptomyces AA9 lytic polysaccharide monooxygenase, or a Gram-negative bacterial polypeptide such as a Campylobacter, E. coli, Flavobacterium, Fusobacterium, Helicobacter, Ilyobacter, Neisseria, Pseudomonas, Salmonella, or Ureaplasma AA9 lytic polysaccharide monooxygenase.

In one embodiment, the AA9 lytic polysaccharide monooxygenase is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis AA9 lytic polysaccharide monooxygenase.

The AA9 lytic polysaccharide monooxygenase may be a fungal AA9 lytic polysaccharide monooxygenase. For example, the AA9 lytic polysaccharide monooxygenase may be a yeast AA9 lytic polysaccharide monooxygenase such as a Candida, Kluyveromyces, Pichia, Saccharomyces, Schizosaccharomyces, or Yarrowia AA9 lytic polysaccharide monooxygenase; or a filamentous fungal AA9 lytic polysaccharide monooxygenase such as an Acremonium, Acrophialophora, Agaricus, Alternaria, Aspergillus, Aurantiporus, Aureobasidium, Botryospaeria, Bulgaria, Ceriporiopsis, Chaetomium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinus, Lentinula, Leptospaeria, Magnaporthe, Melanocarpus, Malbranchea, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Sporormia, Talaromyces, Thermoascus, Thermomyces, Thielavia, Tolypocladium, Trichoderma, Trichophaea, Verticillium, Valsaria, Volvariella, or Xylaria AA9 lytic polysaccharide monooxygenase.

In another embodiment, the AA9 lytic polysaccharide monooxygenase is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis AA9 lytic polysaccharide monooxygenase.

In another embodiment, the AA9 lytic polysaccharide monooxygenase is an Acremonium cellulolyticus, Acrophialophora fusispora, Aspergillus aculeatus, Aspergillus awamori, Aspergillus foetidus, Aspergillus fumigatus, Aspergillus japonicus, Aspergillus lentulus, Aspergillus nidulans, Aspergillus niger, Aspergillus niveus, Aspergillus oryzae, Aspergillus terreus, Aurantiporus alborubescens, Bulgaria inquinans, Chaetomium thermophilum, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Corynascus sepedonium, Corynascus thermophilus, Fennellia nivea, Fusarium bactridioides, Fusarium cerealis, Fusarium crookwellense, Fusarium culmorum, Fusarium graminearum, Fusarium graminum, Fusarium heterosporum, Fusarium longipes, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Lentinus similis, Malbranchea cinnamomea, Mucor miehei, Myceliophthora thermophila, Neurospora crassa, Penicillium capsulatum, Penicillium emersonii, Penicillium funiculosum, Penicillium pinophilum, Penicillium purpurogenum, Penicillium soppii, Penicillium thomii, Phanerochaete chrysosporium, Sporormia fimetaria, Talaromyces byssochlamydoides, Talaromyces emersonii, Talaromyces leycettanus, Talaromyces stipitatus, Talaromyces thermophilus, Thermoascus aurantiacus, Thermoascus crustaceus, Thermomyces lanuginosus, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma atroviride, Trichoderma harzianum, Trichoderma koningii, Trichoderma longibrachiatum, Trichoderma reesei, Trichoderma saturnisporum, Trichoderma viride, or Valsaria rubricosa AA9 lytic polysaccharide monooxygenase.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The AA9 lytic polysaccharide monooxygenase may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding an AA9 lytic polysaccharide monooxygenase may then be obtained by similarly screening a genomic DNA or cDNA library of another microorganism or mixed DNA sample. Once a polynucleotide encoding an AA9 lytic polysaccharide monooxygenase has been detected with the probe(s), the polynucleotide can be isolated or cloned by utilizing techniques that are known to those of ordinary skill in the art (see, e.g., Sambrook et al., 1989, supra).

In an embodiment, the AA9 lytic polysaccharide monooxygenase constitutes from 0.1-25%, such as 0.5-20%, 0.5-15%, 0.5-10%, or 0.5-7% of the enzyme composition. In another embodiment, the amount of AA9 lytic polysaccharide monooxygenase to the enzyme composition is about 1 g to about 1000 g, such as about 1 g to about 200 g, about 1 g to about 100 g, about 1 g to about 50 g, about 1 g to about 20 g, about 1 g to about 15 g, about 1 g to about 10 g, about 1 g to about 7 g, or about 1 g to about 4 g per g of the enzyme composition.

### Oxidoreductases

In the methods of the present invention, the oxidoreductase may be a catalase, a laccase, a peroxidase, a superoxide dismutase, or a combination thereof.

In one aspect, the one or more added oxidoreductases is a catalase. In another aspect, the one or more added oxidoreductases is a laccase. In another aspect, the one or more added oxidoreductases is a peroxidase. In another aspect, the one or more added oxidoreductases is a superoxide dismutase. In another aspect, the one or more added oxidoreductases is a combination of two or more oxidoreductases selected from the group consisting of a catalase, a laccases, a peroxidase, and a superoxide dismutase.

The catalase may be any catalase useful in the methods of the present invention. The catalase may include, but is not limited to, an E.C. 1.11.1.6 or E.C. 1.11.1.21 catalase.

Examples of useful catalases include, but are not limited to, catalases from Alcaligenes aquamarinus (WO 98/00526), Aspergillus lentulus, Aspergillus fumigatus (Paris et al., 2003, Infect lmmun. 71(6): 3551-3562., Aspergillus niger (U.S. Pat. No. 5,360,901), Aspergillus oryzae (JP2002223772A; U.S. Pat. No. 6,022,721), Bacillus thermoglucosidasius (JP11243961A), Humicola insolens (WO 2009/104622, WO 2012/130120), Malbranchea cinnamomea (US 2014/0335572), Microscilla furvescens (WO 98/00526), Neurospora crassa (Dominguez et al., 2010, Arch. Biochem. Biophys. 500: 82-91), Penicillium emersonii (WO 2012/130120), Penicillium pinophilum (EP2256192), Rhizomucor pusillus (US 2014/0335572), Saccharomyces pastorianus (WO 2007/105350), Scytalidium thermophilum (Sutay Kocabas et al., 2009, Acta Crystallogr. Sect. F 65: 486-488), Talaromyces stipitatus (WO 2012/130120), Thermoascus aurantiacus (WO 2012/130120), Thermus brockianus (WO 2005/044994), and Thielavia terrestris (WO 2010/074972).

Non-limiting examples of catalases useful in the present invention are catalases from Bacillus pseudofirmus (UniProt: P30266), Bacillus subtilis (UniProt: P42234), Humicola grisea (GeneSeqP: AXQ55105), Neosartorya fischeri (UniProt: A1DJU9), Neurospora crassa (UniProt: Q9C168), Penicillium emersonii (GeneSeqP: BAC10987), Penicillium pinophilum (GeneSeqP:BAC10995), Scytalidium thermophilum (GeneSeqP: AAW06109 or GeneSeqP: ADT89624), Talaromyces stipitatus (GeneSeqP: BAC10983 or GeneSeqP: BAC11039; UniProt: B8MT74), and Thermoascus aurantiacus (GeneSeqP: BAC11005; SEQ ID NO: 8). The accession numbers are incorporated herein in their entirety.

The laccase may be any laccase useful in the methods of the present invention. The laccase may include, but is not limited to, an E.C. 1.10.3.2 laccase.

Examples of useful laccases include, but are not limited to, laccases from Coprinus cinereus (WO 97/008325; Schneider et al., 1999, Enzyme and Microbial Technology 25: 502-508), Corynascus thermophilus (WO 2013/087027), Melanocarpus albomyces (Kiiskinen et al., 2004, Microbiology 150: 3065-3074), Myceliophthora thermophila (WO 95/033836, WO 2006/012902), Polyporus pinsitus (WO 96/000290, WO 2014/028833), Polyporus versicolor (Jönsson et al., 1998, Appl. Microbiol. Biotechnol. 49: 691-697), Pycnoporus cinnabarinus, Pyricularia oryzae (Muralikrishna et al., 1995, Appl. Environ. Microbiol. 61(12): 4374-4377), Rhizoctonia solani (WO 95/007988; WO 97/009431; Waleithner et al., 1996, Curr. Genet. 29: 395-403), Rhus vernicifera (Yoshida, 1983, Chemistry of Lacquer (Urushi) part 1. J. Chem. Soc. 43: 472-486), Scytalidium thermophilum (WO 95/033837, WO 97/019999), Streptomyces coelicolor (Machczynski et al., 2004, in Protein Science 13: 2388-2397), and Trametes versicolor (WO 96/000290).

Non-limiting examples of laccases useful in the present invention are laccases from Coprinus cinereus (GeneSeqP: AAW17974, GeneSeqP: AAW17975), Corynascus thermophilus (GeneSeqP: BAP78725), Myceliophthora thermophila (GeneSeqP: AAR88500, GeneSeqP: AEF76888), Polyporus pinsitus (GeneSeqP: BBD26012, GeneSeqP: AAR90721), Rhizoctonia solani (GeneSeqP: AAR72328, GeneSeqP: AAW16301), Scytalidium thermophilum (GeneSeqP: AAR88500, GeneSeqP: AAW19855), and Trametes versicolor (GeneSeqP: AAR90722). The accession numbers are incorporated herein in their entirety.

The peroxidase may be any peroxidase useful in the methods of the present invention. The peroxidase may include, but is not limited to, an E.C. 1.11.1.x peroxidase, e.g., E.C. 1.11.1.1 NADH peroxidase, E.C. 1.11.1.2 NADPH peroxidase, E.C. 1.11.1.3 fatty acid peroxidase, E.C. 1.11.1.5 di-heme cytochrome c peroxidase, E.C. 1.11.1.5 cytochrome c peroxidase, E.C. 1.11.1.6 catalase, E.C. 1.11.1.6 manganese catalase, E.C. 1.11.1.7 invertebrate peroxinectin, E.C. 1.11.1.7 eosinophil peroxidase, E.C. 1.11.1.7 lactoperoxidase, E.C. 1.11.1.7 myeloperoxidase, E.C. 1.11.1.8 thyroid peroxidase, E.C. 1.11.1.9 glutathione peroxidase, E.C. 1.11.1.10 chloride peroxidase, E.C. 1.11.1.11 ascorbate peroxidase, E.C. 1.11.1.12 other glutathione peroxidase, E.C. 1.11.1.13 manganese peroxidase, E.C. 1.11.1.14 lignin peroxidase, E.C. 1.11.1.15 cysteine peroxiredoxin, E.C. 1.11.1.16 versatile peroxidase, E.C. 1.11.1.17 glutathione amide-dependent peroxidase, E.C. 1.11.1.18 bromide peroxidase, E.C. 1.11.1.19 dye decolorizing peroxidase, E.C. 1.11.1.B2 chloride peroxidase, E.C. 1.11.1.B4 haloperoxidase, E.C. 1.11.1.B4 no-heme vanadium haloperoxidase, E.C. 1.11.1.B6 iodide peroxidase, E.C. 1.11.1.B7 bromide peroxidase, and E.C. 1.11.1.B8 iodide peroxidase.

Examples of useful peroxidases include, but are not limited to, Coprinus cinereus peroxidase (Baunsgaard et al., 1993, Eur. J. Biochem. 213 (1): 605-611; WO 92/016634); horseradish peroxidase (Fujiyama et al., 1988, Eur. J. Biochem. 173 (3): 681-687); peroxiredoxin (Singh and Shichi, 1998, J. Biol. Chem. 273 (40): 26171-26178); lactoperoxidase (Dull et al., 1990, DNA Cell Biol. 9 (7): 499-509); eosinophil peroxidase (Fornhem et al., 1996, Int. Arch. Allergy lmmunol. 110 (2): 132-142); versatile peroxidase (Ruiz-Duenas et al., 1999, Mol. Microbiol. 31 (1): 223-235); turnip peroxidase (Mazza and Welinder, 1980, Eur. J. Biochem. 108 (2): 481-489); myeloperoxidase (Morishita et al., 1987, J. Biol. Chem. 262: 15208-15213); peroxidasin and peroxidasin homologs (Horikoshi et al., 1999, Biochem. Biophys. Res. Commun. 261 (3): 864-869); lignin peroxidase (Tien and Tu, 1987, Nature 326 (6112): 520-523); and manganese peroxidase (Orth et al., 1994, Gene 148 (1): 161-165).

Non-limiting examples of peroxidases useful in the present invention are peroxidases from Coprinus cinereus (UniProt: P28314), Bos taurus (UniProt: O77834, UniProt: P80025), Brassica rapa subsp. Rapa (UniProt: P00434), Homo sapiens (UniProt: P05164, UniProt: Q92616), horseradish peroxidase (UniProt: P15232), Pleurotus eryngii (UniProt: O94753), Phanerochaete chrysosporium (UniProt: P06181, UniProt: P78733), and Sus scrofa (UniProt: P80550). The accession numbers are incorporated herein in their entirety.

The superoxide dismutase may be any superoxide dismutase useful in the methods of the present invention. The superoxide dismutase may include, but is not limited to, an E.C. 1.15.1.1 superoxide dismutase.

Examples of useful superoxide dismutases include, but are not limited to, superoxide dismutases from Aspergillus flavus (Holdom et al., 1996, Infect. Immun. 64: 3326-3332), Aspergillus nidulans (Holdom et al., 1996, Infect. Immun. 64: 3326-3332), Aspergillus niger (Dolashki et al., 2008, Spectrochim. Acta A. Mol. Biomol. Spectrosc. 71, 975-983), Aspergillus terreus (Holdom et al., 1996, Infect. Immun. 64: 3326-3332), Bacillus cereus (Wang et al., 2007, FEMS Microbiol. Lett. 272: 206-213), Chaetomium thermophilum (Zhang et al., 2011, Biotechnol. Lett. 33: 1127-1132), Kluyveromyces marxianus (Nedeva et al., 2009, Chromatogr. B 877: 3529-3536), Myceliophthora thermophila (WO 2012/068236), Rasamsonia emersonii (WO 2014/002616), Saccharomyces cerevisiae (Borders et al., 1998, Biochemistry 37, 11323-11331), Talaromyces marneffei (Thirach et al., 2007, Med. Mycol. 45: 409-417), Thermoascus aurantiacus (Shijin et al., 2007, Biosci. Biotechnol. Biochem. 71: 1090-1093; Song et al., 2009, J. Microbiol. 47: 123-130), and Thielavia terrestris (Berka et al., 2011, Nat. Biotechnol. 29: 922-927).

Non-limiting examples of superoxide dismutases useful in the present invention are superoxide dismutases from Bacillus cereus (UniProt: Q6QHT3), Chaetomium thermophilum (UniProt: Q1HEQ0), Kluyveromyces marxianus (UniProt: BOB552), Myceliophthora thermophila (GeneSeqP: AZW56690), Rasamsonia emersonii (GeneSeqP: BBT31699), Talaromyces marneffei (UniProt: B6QEB3), Thermoascus aurantiacus (UniProt: Q1HDV5, UniProt: Q1HDV5), and Thielavia terrestris (UniProt: G2R3V2). The accession numbers are incorporated herein in their entirety.

In one aspect, the oxidoreductase, e.g., catalase, laccase, peroxidase, or superoxide dismutase, has a sequence identity to the mature polypeptide of an oxidoreductase disclosed herein of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, which has oxidoreductase activity.

In another aspect, the amino acid sequence of the oxidoreductase, e.g., catalase, laccase, peroxidase, or superoxide dismutase, differs by up to 10 amino acids, e.g., 1, 2, 3, 4, 5, 6, 7, 8, 9, or 10 from the mature polypeptide of an oxidoreductase disclosed herein.

In another aspect, the oxidoreductase, e.g., catalase, laccase, peroxidase, or superoxide dismutase, comprises or consists of the amino acid sequence of an oxidoreductase disclosed herein.

In another aspect, the oxidoreductase, e.g., catalase, laccase, peroxidase, or superoxide dismutase, comprises or consists of the mature polypeptide of an oxidoreductase disclosed herein.

In another embodiment, the oxidoreductase, e.g., catalase, laccase, peroxidase, or superoxide dismutase, is an allelic variant of an oxidoreductase disclosed herein.

In another aspect, the oxidoreductase, e.g., catalase, laccase, peroxidase, or superoxide dismutase, is a fragment containing at least 85% of the amino acid residues, e.g., at least 90% of the amino acid residues or at least 95% of the amino acid residues of the mature polypeptide of an oxidoreductase disclosed herein.

In another aspect, the oxidoreductase, e.g., catalase, laccase, peroxidase, or superoxide dismutase, is encoded by a polynucleotide that hybridizes under very low, low, medium, medium-high, high, or very high stringency conditions with the mature polypeptide coding sequence or the full-length complement thereof of an oxidoreductase disclosed herein (Sambrook et al., 1989, supra).

The polynucleotide encoding an oxidoreductase, or a subsequence thereof, as well as the polypeptide of an oxidoreductase, or a fragment thereof, may be used to design nucleic acid probes to identify and clone DNA encoding an oxidoreductase from strains of different genera or species according to methods well known in the art. In particular, such probes can be used for hybridization with the genomic DNA or cDNA of a cell of interest, as described supra.

For purposes of the present invention, hybridization indicates that the polynucleotide hybridizes to a labeled nucleic acid probe under very low to very high stringency conditions. Molecules to which the nucleic acid probe hybridizes under these conditions can be detected using, for example, X-ray film or any other detection means known in the art.

In one aspect, the nucleic acid probe is the mature polypeptide coding sequence of an oxidoreductase.

In another aspect, the nucleic acid probe is a polynucleotide that encodes a full-length oxidoreductase; the mature polypeptide thereof; or a fragment thereof.

In another aspect, the oxidoreductase, e.g., catalase, laccase, peroxidase, or superoxide dismutase, is encoded by a polynucleotide having a sequence identity to the mature polypeptide coding sequence of an oxidoreductase disclosed herein of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

The oxidoreductase, e.g., catalase, laccase, peroxidase, or superoxide dismutase, may be a hybrid polypeptide in which a region of one polypeptide is fused at the N-terminus or the C-terminus of a region of another polypeptide or a fusion polypeptide or cleavable fusion polypeptide in which another polypeptide is fused at the N-terminus or the C-terminus of the oxidoreductase, as described herein.

The protein content of the added oxidoreductase, e.g., catalase, laccase, peroxidase, or superoxide dismutase, is in the range of about 0.1% to about 10%, e.g., about 0.1% to about 7%, about 0.1% to about 5%, about 0.1% to about 4%, about 0.1% to about 3%, about 0.1% to about 2%, and about 0.1% to about 1% of total enzyme protein in the enzyme composition. In an embodiment, the protein ratio of the added oxidoreductase, e.g., catalase, laccase, peroxidase, or superoxide dismutase, to the AA9 lytic polysaccharide monooxygenase is in the range of about 1:250 to about 1:10, e.g., about 1:200 to about 1:10, about 1:150 to about 1:15, about 1:100 to about 1:15, about 1:75 to about 1:20, or about 1:50 to about 1:25.

### Host Cells

In the methods of present invention, the host cell can be a wild-type host cell or a recombinant host cell. The term “host cell” encompasses any progeny of a parent cell that is not identical to the parent cell due to mutations that occur during replication.

The host cell may be any cell useful in the production of an enzyme composition. In one aspect, the host cell is a prokaryote. In another aspect, the host cell is a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Enzyme Compositions

The enzyme compositions can comprise one or more (e.g., several) enzymes selected from the group consisting of a hydrolase, an isomerase, a ligase, a lyase, an oxidoreductase, or a transferase.

In one aspect, the enzyme compositions can comprise one or more (e.g., several) enzymes selected from the group consisting of an alpha-galactosidase, an alpha-glucosidase, an aminopeptidase, an amylase, a beta-galactosidase, a beta-glucosidase, a beta-xylosidase, a carbohydrase, a carboxypeptidase, a catalase, a cellobiohydrolase, a cellulase, a chitinase, a cutinase, a cyclodextrin glycosyltransferase, a deoxyribonuclease, an endoglucanase, an esterase, a glucoamylase, an invertase, a laccase, a lipase, a mannosidase, a mutanase, an oxidase, a pectinolytic enzyme, a peroxidase, a phytase, a polyphenoloxidase, a proteolytic enzyme, a ribonuclease, a transglutaminase, and a xylanase.

In another aspect, the enzyme compositions can comprise any protein useful in degrading a lignocellulosic material, e.g., cellulosic or hemicellulosic material.

In another aspect, the enzyme composition comprises or further comprises one or more (e.g., several) proteins selected from the group consisting of a cellulase, an AA9 polypeptide, a hemicellulase, a cellulose inducing protein (CIP), an esterase, an expansin, a ligninolytic enzyme, a pectinase, a protease, and a swollenin. In another aspect, the cellulase is preferably one or more (e.g., several) enzymes selected from the group consisting of an endoglucanase, a cellobiohydrolase, and a beta-glucosidase. In another aspect, the hemicellulase is preferably one or more (e.g., several) enzymes selected from the group consisting of an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase.

In another aspect, the enzyme composition comprises one or more (e.g., several) cellulolytic enzymes. In another aspect, the enzyme composition comprises or further comprises one or more (e.g., several) hemicellulolytic enzymes. In another aspect, the enzyme composition comprises one or more (e.g., several) cellulolytic enzymes and one or more (e.g., several) hemicellulolytic enzymes. In another aspect, the enzyme composition comprises one or more (e.g., several) enzymes selected from the group of cellulolytic enzymes and hemicellulolytic enzymes. In another aspect, the enzyme composition comprises an endoglucanase. In another aspect, the enzyme composition comprises a cellobiohydrolase. In another aspect, the enzyme composition comprises a beta-glucosidase. In another aspect, the enzyme composition comprises an AA9 polypeptide. In another aspect, the enzyme composition comprises an endoglucanase and an AA9 polypeptide. In another aspect, the enzyme composition comprises a cellobiohydrolase and an AA9 polypeptide. In another aspect, the enzyme composition comprises a beta-glucosidase and an AA9 polypeptide. In another aspect, the enzyme composition comprises an endoglucanase and a cellobiohydrolase. In another aspect, the enzyme composition comprises an endoglucanase I, an endoglucanase II, or a combination of an endoglucanase I and an endoglucanase II, and a cellobiohydrolase I, a cellobiohydrolase II, or a combination of a cellobiohydrolase I and a cellobiohydrolase II. In another aspect, the enzyme composition comprises an endoglucanase and a beta-glucosidase. In another aspect, the enzyme composition comprises an endoglucanase I, an endoglucanase II, or a combination of an endoglucanase I and an endoglucanase II, and a beta-glucosidase. In another aspect, the enzyme composition comprises a beta-glucosidase and a cellobiohydrolase. In another aspect, the enzyme composition comprises a beta-glucosidase and a cellobiohydrolase I, a cellobiohydrolase II, or a combination of a cellobiohydrolase I and a cellobiohydrolase II. In another aspect, the enzyme composition comprises an endoglucanase, an AA9 polypeptide, and a cellobiohydrolase. In another aspect, the enzyme composition comprises an endoglucanase I, an endoglucanase II, or a combination of an endoglucanase I and an endoglucanase II, an AA9 polypeptide, and a cellobiohydrolase I, a cellobiohydrolase II, or a combination of a cellobiohydrolase I and a cellobiohydrolase II. In another aspect, the enzyme composition comprises an endoglucanase, a beta-glucosidase, and an AA9 polypeptide. In another aspect, the enzyme composition comprises a beta-glucosidase, an AA9 polypeptide, and a cellobiohydrolase. In another aspect, the enzyme composition comprises a beta-glucosidase, an AA9 polypeptide, and a cellobiohydrolase I, a cellobiohydrolase II, or a combination of a cellobiohydrolase I and a cellobiohydrolase II. In another aspect, the enzyme composition comprises an endoglucanase, a beta-glucosidase, and a cellobiohydrolase. In another aspect, the enzyme composition comprises an endoglucanase I, an endoglucanase II, or a combination of an endoglucanase I and an endoglucanase II, a beta-glucosidase, and a cellobiohydrolase I, a cellobiohydrolase II, or a combination of a cellobiohydrolase I and a cellobiohydrolase II. In another aspect, the enzyme composition comprises an endoglucanase, a cellobiohydrolase, a beta-glucosidase, and an AA9 polypeptide. In another aspect, the enzyme composition comprises an endoglucanase I, an endoglucanase II, or a combination of an endoglucanase I and an endoglucanase II, a beta-glucosidase, an AA9 polypeptide, and a cellobiohydrolase I, a cellobiohydrolase II, or a combination of a cellobiohydrolase I and a cellobiohydrolase II.

In another aspect, the enzyme composition comprises an acetylmannan esterase. In another aspect, the enzyme composition comprises an acetylxylan esterase. In another aspect, the enzyme composition comprises an arabinanase (e.g., alpha-L-arabinanase). In another aspect, the enzyme composition comprises an arabinofuranosidase (e.g., alpha-L-arabinofuranosidase). In another aspect, the enzyme composition comprises a coumaric acid esterase. In another aspect, the enzyme composition comprises a feruloyl esterase. In another aspect, the enzyme composition comprises a galactosidase (e.g., alpha-galactosidase and/or beta-galactosidase). In another aspect, the enzyme composition comprises a glucuronidase (e.g., alpha-D-glucuronidase). In another aspect, the enzyme composition comprises a glucuronoyl esterase. In another aspect, the enzyme composition comprises a mannanase. In another aspect, the enzyme composition comprises a mannosidase (e.g., beta-mannosidase). In another aspect, the enzyme composition comprises a xylanase. In an embodiment, the xylanase is a Family 10 xylanase. In another embodiment, the xylanase is a Family 11 xylanase. In another aspect, the enzyme composition comprises a xylosidase (e.g., beta-xylosidase).

In another aspect, the enzyme composition comprises an esterase. In another aspect, the enzyme composition comprises an expansin. In another aspect, the enzyme composition comprises a ligninolytic enzyme. In an embodiment, the ligninolytic enzyme is a manganese peroxidase. In another embodiment, the ligninolytic enzyme is a lignin peroxidase. In another embodiment, the ligninolytic enzyme is a H₂O₂-producing enzyme. In another aspect, the enzyme composition comprises a pectinase. In another aspect, the enzyme composition comprises an oxidoreductase. In another aspect, the enzyme composition comprises a protease. In another aspect, the enzyme composition comprises a swollenin.

One or more (e.g., several) components of the enzyme composition may be native proteins, recombinant proteins, or a combination of native proteins and recombinant proteins. For example, one or more (e.g., several) components may be native proteins of a cell, which is used as a host cell to express recombinantly one or more (e.g., several) other components of the enzyme composition. It is understood herein that the recombinant proteins may be heterologous (e.g., foreign) and/or native to the host cell. One or more (e.g., several) components of the enzyme composition may be produced as monocomponents, which are then combined to form the enzyme composition. The enzyme composition may be a combination of multicomponent and monocomponent protein preparations.

The polypeptides having cellulolytic enzyme activity or hemicellulolytic enzyme activity as well as other proteins/polypeptides useful in the degradation of the cellulosic or hemicellulosic material, e.g., AA9 polypeptides can be derived or obtained from any suitable origin, including, archaeal, bacterial, fungal, yeast, plant, or animal origin. The term “obtained” also means herein that the enzyme may have been produced recombinantly in a host organism employing methods described herein, wherein the recombinantly produced enzyme is either native or foreign to the host organism or has a modified amino acid sequence, e.g., having one or more (e.g., several) amino acids that are deleted, inserted and/or substituted, i.e., a recombinantly produced enzyme that is a mutant and/or a fragment of a native amino acid sequence or an enzyme produced by nucleic acid shuffling processes known in the art. Encompassed within the meaning of a native enzyme are natural variants and within the meaning of a foreign enzyme are variants obtained by, e.g., site-directed mutagenesis or shuffling.

Each polypeptide may be a bacterial polypeptide. For example, each polypeptide may be a Gram-positive bacterial polypeptide having enzyme activity, or a Gram-negative bacterial polypeptide having enzyme activity.

Each polypeptide may also be a fungal polypeptide, e.g., a yeast polypeptide or a filamentous fungal polypeptide.

Chemically modified or protein engineered mutants of polypeptides may also be used.

One or more (e.g., several) components of the enzyme composition may be a recombinant component, i.e., produced by cloning of a DNA sequence encoding the single component and subsequent cell transformed with the DNA sequence and expressed in a host (see, for example, WO 91/17243 and WO 91/17244). The host can be a heterologous host (enzyme is foreign to host), but the host may under certain conditions also be a homologous host (enzyme is native to host). Monocomponent cellulolytic proteins may also be prepared by purifying such a protein from a fermentation broth.

In one aspect, the one or more (e.g., several) cellulolytic enzymes comprise a commercial cellulolytic enzyme preparation. Examples of commercial cellulolytic enzyme preparations suitable for use in the present invention include, for example, CELLIC® CTec (Novozymes A/S), CELLIC® CTec2 (Novozymes A/S), CELLIC® CTec3 (Novozymes A/S), CELLUCLAST™ (Novozymes A/S), NOVOZYM™ 188 (Novozymes A/S), SPEZYME™ CP (Genencor Int.), ACCELLERASE™ TRIO (DuPont), FILTRASE® NL (DSM); METHAPLUS® S/L 100 (DSM), ROHAMENT™ 7069 W (Röhm GmbH), or ALTERNAFUEL® CMAX3™ (Dyadic International, Inc.). The cellulolytic enzyme preparation is added in an amount effective from about 0.001 to about 5.0 wt. % of solids, e.g., about 0.025 to about 4.0 wt. % of solids or about 0.005 to about 2.0 wt. % of solids.

Examples of bacterial endoglucanases include, but are not limited to, Acidothermus cellulolyticus endoglucanase (WO 91/05039; WO 93/15186; U.S. Pat. No. 5,275,944; WO 96/02551; U.S. Pat. No. 5,536,655; WO 00/70031; WO 05/093050), Erwinia carotovara endoglucanase (Saarilahti et al., 1990, Gene 90: 9-14), Thermobifida fusca endoglucanase III (WO 05/093050), and Thermobifida fusca endoglucanase V (WO 05/093050).

Examples of fungal endoglucanases that can be used in the present invention, include, but are not limited to, Trichoderma reesei endoglucanase I (Penttila et al., 1986, Gene 45: 253-263, Trichoderma reesei Cel7B endoglucanase I (GenBank:M15665), Trichoderma reesei endoglucanase II (Saloheimo et al., 1988, Gene 63:11-22), Trichoderma reesei Cel5A endoglucanase II (GenBank:M19373), Trichoderma reesei endoglucanase III (Okada et al., 1988, Appl. Environ. Microbiol. 64: 555-563, GenBank:AB003694), Trichoderma reesei endoglucanase V (Saloheimo et al., 1994, Molecular Microbiology 13: 219-228, GenBank:Z33381), Aspergillus aculeatus endoglucanase (Ooi et al., 1990, Nucleic Acids Research 18: 5884), Aspergillus kawachii endoglucanase (Sakamoto et al., 1995, Current Genetics 27: 435-439), Fusarium oxysporum endoglucanase (GenBank:L29381), Humicola grisea var. thermoidea endoglucanase (GenBank:AB003107), Melanocarpus albomyces endoglucanase (GenBank:MAL515703), Neurospora crassa endoglucanase (GenBank:XM_324477), Humicola insolens endoglucanase V, Myceliophthora thermophila CBS 117.65 endoglucanase, Thermoascus aurantiacus endoglucanase I (GenBank:AF487830), Trichoderma reesei strain No. VTT-D-80133 endoglucanase (GenBank:M15665), and Penicillium pinophilum endoglucanase (WO 2012/062220).

Examples of cellobiohydrolases useful in the present invention include, but are not limited to, Aspergillus aculeatus cellobiohydrolase II (WO 2011/059740), Aspergillus fumigatus cellobiohydrolase I (WO 2013/028928), Aspergillus fumigatus cellobiohydrolase II (WO 2013/028928), Chaetomium thermophilum cellobiohydrolase I, Chaetomium thermophilum cellobiohydrolase II, Humicola insolens cellobiohydrolase I, Myceliophthora thermophila cellobiohydrolase II (WO 2009/042871), Penicillium occitanis cellobiohydrolase I (GenBank:AY690482), Talaromyces emersonii cellobiohydrolase I (Gen Bank:AF439936), Thielavia hyrcanie cellobiohydrolase II (WO 2010/141325), Thielavia terrestris cellobiohydrolase II (CEL6A, WO 2006/074435), Trichoderma reesei cellobiohydrolase I, Trichoderma reesei cellobiohydrolase II, and Trichophaea saccata cellobiohydrolase II (WO 2010/057086).

Examples of beta-glucosidases useful in the present invention include, but are not limited to, beta-glucosidases from Aspergillus aculeatus (Kawaguchi et al., 1996, Gene 173: 287-288), Aspergillus fumigatus (WO 2005/047499), Aspergillus niger (Dan et al., 2000, J. Biol. Chem. 275: 4973-4980), Aspergillus oryzae (WO 02/095014), Penicillium brasilianum IBT 20888 (WO 2007/019442 and WO 2010/088387), Thielavia terrestris (WO 2011/035029), and Trichophaea saccata (WO 2007/019442).

Other useful endoglucanases, cellobiohydrolases, and beta-glucosidases are disclosed in numerous Glycosyl Hydrolase families using the classification according to Henrissat, 1991, Biochem. J. 280: 309-316, and Henrissat and Bairoch, 1996, Biochem. J. 316: 695-696.

In one aspect, the one or more (e.g., several) hemicellulolytic enzymes comprise a commercial hemicellulolytic enzyme preparation. Examples of commercial hemicellulolytic enzyme preparations suitable for use in the present invention include, for example, SHEARZYME™ (Novozymes A/S), CELLIC® HTec (Novozymes A/S), CELLIC® HTec2 (Novozymes A/S), CELLIC® HTec3 (Novozymes A/S), VISCOZYME® (Novozymes A/S), ULTRAFLO® (Novozymes A/S), PULPZYME® HC (Novozymes A/S), MULTIFECT® Xylanase (Genencor), ACCELLERASE® XY (Genencor), ACCELLERASE® XC (Genencor), ECOPULP® TX-200A (AB Enzymes), HSP 6000 Xylanase (DSM), DEPOL™ 333P (Biocatalysts Limit, Wales, UK), DEPOL™ 740L. (Biocatalysts Limit, Wales, UK), and DEPOL™ 762P (Biocatalysts Limit, Wales, UK), ALTERNA FUEL 100P (Dyadic), and ALTERNA FUEL 200P (Dyadic).

Examples of xylanases include, but are not limited to, xylanases from Aspergillus aculeatus (GeneSeqP:AAR63790; WO 94/21785), Aspergillus fumigatus (WO 2006/078256), Penicillium pinophilum (WO 2011/041405) , Penicillium sp. (WO 2010/126772), Thermomyces lanuginosus (GeneSeqP:BAA22485), Talaromyces thermophilus (GeneSeqP:BAA22834), Thielavia terrestris NRRL 8126 (WO 2009/079210), and Trichophaea saccata (WO 2011/057083).

Examples of beta-xylosidases include, but are not limited to, beta-xylosidases from Neurospora crassa (Swiss Prot:Q7SOW4), Trichoderma reesei (UniProtKB/TrEMBL:Q92458), Talaromyces emersonii (SwissProt:Q8X212), and Talaromyces thermophilus (GeneSeqP:BAA22816).

Examples of acetylxylan esterases include, but are not limited to, acetylxylan esterases from Aspergillus aculeatus (WO 2010/108918), Chaetomium globosum (UniProt:Q2GWX4), Chaetomium gracile (GeneSeqP:AAB82124), Humicola insolens DSM 1800 (WO 2009/073709), Hypocrea jecorina (WO 2005/001036), Myceliophtera thermophila (WO 2010/014880), Neurospora crassa (UniProt:q7s259), Phaeosphaeria nodorum (UniProt:Q0UHJ1), and Thielavia terrestris NRRL 8126 (WO 2009/042846).

Examples of feruloyl esterases (ferulic acid esterases) include, but are not limited to, feruloyl esterases form Humicola insolens DSM 1800 (WO 2009/076122), Neosartorya fischeri (UniProt:A1D9T4), Neurospora crassa (UniProt:Q9HGR3), Penicillium aurantiogriseum (WO 2009/127729), and Thielavia terrestris (WO 2010/053838 and WO 2010/065448).

Examples of arabinofuranosidases include, but are not limited to, arabinofuranosidases from Aspergillus niger (GeneSeqP:AAR94170), Humicola insolens DSM 1800 (WO 2006/114094 and WO 2009/073383), and M. giganteus (WO 2006/114094).

Examples of alpha-glucuronidases include, but are not limited to, alpha-glucuronidases from Aspergillus clavatus (UniProt:alcc12), Aspergillus fumigatus (SwissProt:Q4WW45), Aspergillus niger (UniProt:Q96WX9), Aspergillus terreus (SwissProt:Q0CJ P9), Humicola insolens (WO 2010/014706), Penicillium aurantiogriseum (WO 2009/068565), Talaromyces emersonii (UniProt:Q8X211), and Trichoderma reesei (UniProt:Q99024).

In one aspect, the oxidoreductase, e.g., catalase, laccase, peroxidase, and superoxide dismutase, inhibits AA9 lytic polysaccharide monooxygenase catalyzed inactivation of an enzyme composition or a component thereof. In one aspect, the enzyme component is a cellulase. In another aspect, the enzyme component is a hemicellulase. In another aspect, the enzyme component is a cellulose inducing protein (CIP). In another aspect, the enzyme component is an esterase. In another aspect, the enzyme component is an expansin. In another aspect, the enzyme component is a ligninolytic enzyme. In another aspect, the enzyme component is a pectinase. In another aspect, the enzyme component is a protease. In another aspect, the enzyme component is a swollenin. In another aspect, the enzyme component is a cellobiohydrolase. In another aspect, the enzyme component is a cellobiohydrolase I. In another aspect, the enzyme component is a cellobiohydrolase II. In another aspect, the enzyme component is an endoglucanase. In another aspect, the enzyme component is a beta-glucosidase. In another aspect, the enzyme component is a xylanase. In another aspect, the enzyme component is a beta-xylosidase.

The composition components may be produced by fermentation of the above-noted host cells on a nutrient medium containing suitable carbon and nitrogen sources and inorganic salts, using procedures known in the art (see, e.g., Bennett, J. W. and LaSure, L. (eds.), More Gene Manipulations in Fungi, Academic Press, CA, 1991). Suitable media are available from commercial suppliers or may be prepared according to published compositions (e.g., in catalogues of the American Type Culture Collection). Temperature ranges and other conditions suitable for growth and enzyme production are known in the art (see, e.g., Bailey, J. E., and Ollis, D. F., Biochemical Engineering Fundamentals, McGraw-Hill Book Company, NY, 1986).

The fermentation can be any method of cultivation of a cell resulting in the expression or isolation of an enzyme or protein. Fermentation may, therefore, be understood as comprising shake flask cultivation, or small- or large-scale fermentation (including continuous, batch, fed-batch, or solid state fermentations) in laboratory or industrial fermentors performed in a suitable medium and under conditions allowing the enzyme to be expressed or isolated. The resulting enzymes produced by the methods described above may be recovered from the fermentation medium and purified by conventional procedures.

The enzyme compositions may be in any form suitable for use, such as, for example, a fermentation broth formulation or a cell composition, a cell lysate with or without cellular debris, a semi-purified or purified enzyme preparation, or a host cell as a source of the enzymes. The enzyme composition may be a dry powder or granulate, a non-dusting granulate, a liquid, a stabilized liquid, or a stabilized protected enzyme. Liquid enzyme preparations may, for instance, be stabilized by adding stabilizers such as a sugar, a sugar alcohol or another polyol, and/or lactic acid or another organic acid according to established processes.

The enzyme compositions can be a fermentation broth formulation or a cell composition comprising a polypeptide of the present invention. The fermentation broth product further comprises additional ingredients used in the fermentation process, such as, for example, cells (including, the host cells containing the gene encoding the polypeptide of the present invention which are used to produce the polypeptide), cell debris, biomass, fermentation media and/or fermentation products. In some embodiments, the composition is a cell-killed whole broth containing organic acid(s), killed cells and/or cell debris, and culture medium.

The term “fermentation broth” refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The fermentation broth formulations or cell compositions may further comprise multiple enzymatic activities, such as one or more (e.g., several) enzymes selected from the group consisting of a cellulase, a hemicellulase, an AA9 polypeptide, a cellulose inducible protein (CIP), a catalase, an esterase, an expansin, a laccase, a ligninolytic enzyme, a pectinase, a peroxidase, a protease, and a swollenin. The fermentation broth formulations or cell compositions may also comprise one or more (e.g., several) enzymes selected from the group consisting of a hydrolase, an isomerase, a ligase, a lyase, an oxidoreductase, or a transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis (e.g., expression of cellulase and/or glucosidase enzyme(s)). In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by the method described in WO 90/15861 or WO 2010/096673.

The present invention also relates to a composition comprising an AA9 lytic polysaccharide monooxygenase and one or more added oxidoreductases selected from the group consisting of a catalase, a laccases, a peroxidase, and a superoxide dismutase, wherein the protein ratio of the added oxidoreductase to the AA9 lytic polysaccharide monooxygenase is in the range of about 1:250 to about 1:10, e.g., about 1:200 to about 1:10, about 1:150 to about 1:15, about 1:100 to about 1:15, about 1:75 to about 1:20, or about 1:50 to about 1:25.

The present invention is further described by the following examples that should not be construed as limiting the scope of the invention.

### EXAMPLES

### Strains

Trichoderma reesei strain RutC30 is a mutagenized T. reesei strain of original isolate QM6A (Montenecourt and Eveleigh, 1979, Adv. Chem. Ser. 181: 289-301).

T. reesei strain BTR213 (O326PT) is a mutagenized strain of T. reesei RutC30.

T. reesei strain 981-O8-D4 is a mutagenized strain of T. reesei RutC30.

T. reesei strain BTR-TI12-10 is T. reesei strain BTR213 comprising a replacement of the native cellobiohydrolase I coding sequence with the coding sequence for the cellobiohydrolase I of SEQ ID NO: 2 and a replacement of the native cellobiohydrolase II coding sequence with the coding sequence for the cellobiohydrolase II of SEQ ID NO: 4.

T. reesei strain JfyS99-19B4 is T. reesei strain 981-O8-D4 comprising a replacement of the native cellobiohydrolase I coding sequence with the coding sequence for the cellobiohydrolase I of SEQ ID NO: 2 and a replacement of the native cellobiohydrolase II coding sequence with the coding sequence for the cellobiohydrolase II of SEQ ID NO: 4.

Strain A (T. reesei Q2B-1, O62J7Z) is T. reesei BTR-TI12-10 strain comprising the coding sequence for the AA9 polypeptide of SEQ ID NO: 6.

Strain B (T. reesei AgJg005-35A, O622QV) is T. reesei strain BTR213-TI12-10 comprising the coding sequences for the AA9 polypeptide of SEQ ID NO: 6 and catalase of SEQ ID NO: 8.

Strain C (T. reesei QMJi051-8B-4, O428DH) is T. reesei strain JfyS99-19B4 comprising the coding sequence coding sequence for the AA9 polypeptide of SEQ ID NO: 6.

Strain D (T. reesei AgJg004-202A4, O422W5) is T. reesei strain JfyS99-19B4 comprising the coding sequences for the AA9 polypeptide of SEQ ID NO: 6 and the catalase of SEQ ID NO: 8.

### Media

Fermentation batch medium was composed per liter of 24 g of dextrose, 40 g of soy meal, 8 g of (NH₄)₂SO₄, 3 g of K₂HPO₄, 8 g of K₂SO₄, 3 g of CaCO₃, 8 g of MgSO₄.7H₂O, 1 g of citric acid, 8.8 ml of 85% phosphoric acid, 1 ml of anti-foam, and 14.7 ml of trace metals solution.

PDA plates were composed of 39 g of Potato Dextrose Agar (Difco) and deionized water to 1 liter.

Shake flask medium was composed per liter of 20 g of glycerol, 10 g of soy meal, 1.5 g of (NH₄)₂SO₄, 2 g of KH₂PO₄, 0.2 g of CaCl₂, 0.4 g of MgSO₄.7H₂O, and 0.2 ml of trace metals solution.

Trace metals solution was composed per liter of 26.1 g of FeSO₄.7H₂O, 5.5 g of ZnSO₄.7H₂O, 6.6 g of MnSO₄.H₂O, 2.6 g of CuSO₄.5H₂O and 2 g of citric acid.

### Example 1

### Co-Culture Fermentations of Strains A and B at pH 4.5

Strains A and B were each grown on PDA plates for 4-7 days at 28° C. For each strain, three 500 ml shake flasks each containing 100 ml of shake flask medium were inoculated with two plugs from the respective PDA plate. The shake flasks were incubated at 28° C. for 48 hours on an orbital shaker at 200 rpm. The cultures were used as seeds for larger scale fermentation.

A total of 150 ml of the seed cultures was used to inoculate three liter glass jacketed fermentors (Applikon Biotechnology) each containing 1.5 liters of the fermentation batch medium according to Table 1 below.

TABLE 1
Fermentation at pH 4.5 with several levels of
catalase-expressing strain in co-culture.

| Fermentation | Fermentation pH | Seed A | Seed B |
|---|---|---|---|
| 1 | 4.5 | 100% | 0% |
| 3 | 4.5 | 95% | 5% |
| 5 | 4.5 | 90% | 10% |
| 7 | 4.5 | 75% | 25% |

The fermentors were maintained at a temperature of 28° C. and pH was controlled using a 1030 Bio Controller (Applikon Biotechnology) to a set-point of 4.5+/−0.1. Air was added to the vessel at a rate of 2.5 L/min and the broth was agitated by Rushton impeller rotating at 1100 rpm. Fermentation feed medium composed of dextrose and phosphoric acid was dosed at a rate of 0 to 10 g/L/hour for a period of 165 hours. Daily samples of 1 ml were taken and centrifuged, and the supernatants were stored at −20° C. until Western blot analysis (see Example 10). At the end of the fermentation, whole broth was harvested from the fermentors and centrifuged at 3000×g to remove the biomass. The supernatants were filtered using 0.22 μm SteriTop® filters (Millipore). The filtered supernatants (“filtrates”) were stored at 5-10° C. The protein concentration of the filtrates was determined using a Microplate BCA™ Protein Assay Kit (Thermo Fischer Scientific) in which bovine serum albumin was used as a protein standard. The composition of the filtrates was supplemented before assay by replacement of the filtrate protein with purified beta-glucosidase of SEQ ID NO: 10, GH10 xylanase of SEQ ID NO: 12, and beta-xylosidase of SEQ ID NO: 14 at 5%, 5%, and 3% of total protein, respectively, which resulted in mixtures 1, 3, 5, and 7.

### Example 2

### Co-Culture Fermentations of Strains A and B at pH 3.5

Example 1 was repeated except the pH was controlled to a set-point of 3.5+/−0.1 and the fermentations were inoculated with the seed cultures of Strains A and B according to Table 2 below.

TABLE 2
Fermentation at 3.5 with several levels of
catalase-expressing strain in co-culture.

| Fermentation | Fermentation pH | Seed A | Seed B |
|---|---|---|---|
| 2 | 3.5 | 100% | 0% |
| 4 | 3.5 | 95% | 5% |
| 6 | 3.5 | 90% | 10% |
| 8 | 3.5 | 75% | 25% |

Daily samples of 1 ml were taken and centrifuged, and the supernatants were stored at −20° C. At the end of the fermentations, whole broth was harvested from the fermentors and centrifuged at 3000×g to remove the biomass. The supernatants were filtered using 0.22 μm SteriTop® filters. The filtered supernatants (“filtrates”) were stored at 5 to 10° C. The protein concentration of the filtrates was determined using a Microplate BCA™ Protein Assay Kit in which bovine serum albumin was used as a protein standard. The composition of these filtrates was supplemented before assay by purified beta-glucosidase of SEQ ID NO: 10, GH10 xylanase of SEQ ID NO: 12, and beta-xylosidase of SEQ ID NO: 14 at 5%, 5%, and 3% of total protein, respectively, which resulted in mixtures 2, 4, 6 and 8.

### Example 3

### Preparation of a Catalase Bolus

Terminox® Supreme (Novozymes A/S, Denmark; Lot # ODN00025), a product containing catalase of SEQ ID NO: 8, was desalted in two aliquots of 100 ml on a 550 ml Sephadex G-25 (GE LifeSciences) column in water. The resulting eluted protein peak detected by absorbance at 280 nm was pooled, sterile filtered using 0.22 μm SteriTop® filters, and stored at 4° C. until use. A sample of the filtered pool was desalted using Econo-Pac® 10DG columns (Bio-Rad Laboratories, Inc.). The protein concentration was determined to be 8.7 mg of protein (at least 60% is catalase) per ml using a Microplate BCA™ Protein Assay Kit in which bovine serum albumin was used as a protein standard. The catalase is designated herein as “TS Catalase”.

### Example 4

### Fermentation of Strain D at pH 5.0

Similar to the fermentation in Example 1, but in a fermentor of 2.5 cubic meters, with scaled quantities of batch and feed media, Strain D was fermented at pH 5.0. The resulting broth was centrifuged, filtered, concentrated by evaporation, and admixed with sodium benzoate, sorbate, and glucose. This material was desalted by tangential flow with water using a Vivaflow 200 cartridge with a 10,000 MWCO (Sartorius AG) to remove the sodium benzoate, sorbate and glucose. The resulting desalted concentrate was pooled based on absorbance at 280 nm. HPLC analysis of residual glucose in the desalted pool showed the glucose concentration to be 2.3 mg/ml. The pool was sterile filtered using 0.22 μm SteriTop® filters and stored at 4° C. until use. An aliquot was desalted using Econo-Pac 10DG columns. The protein concentration was determined to be 177 mg of protein per ml using a Microplate BCA™ Protein Assay Kit in which bovine serum albumin was used as a protein standard. The catalase is designated herein as “TRIRE Catalase”.

### Example 5

### Fermentation of Strain C at pH 3.5 and 4.5

Strain C was grown on a PDA plate for 4-7 days at 28° C. Three 500 ml shake flasks each containing 100 ml of shake flask medium were inoculated with two plugs from the solid plate culture and incubated at 28° C. for 48 hours on an orbital shaker at 200 rpm. This step was repeated to produce sufficient seed culture for 5 fermentors (fermentations 9-13). The cultures were used as seeds for larger scale fermentation.

A total of 150 ml of the Strain C seed culture was used to inoculate three liter glass jacketed fermentors (Applikon Biotechnology) each containing 1.5 liters of fermentation batch medium supplemented with catalase protein (Examples 3 and 4) according to Table 3 below.

TABLE 3

| | | | TRIRE | TS |
|---|---|---|---|---|
| Fermentation | Fermentation pH | Seed C | Catalase | Catalase |
| 9 | 3.5 | 100% | | |
| 10 | 4.5 | 100% | | |
| 11 | 4.5 | 100% | | 113 ml |
| 12 | 4.5 | 100% | 113 ml | |
| 13 | 3.5 | 100% | | |

The fermentors were maintained at a temperature of 28° C. and pH was controlled using a 1030 Bio Controller (Applikon Biotechnology) to a set-point of 4.5 or 3.5+/−0.1. Air was added to the fermentors at a rate of 2.5 L/min and the broth was agitated by Rushton impeller rotating at 1100 rpm. Fermentation feed medium composed of dextrose and phosphoric acid was dosed at a rate of 0 to 10 g/L/hour for a period of 165 hours. At the end of the fermentation, whole broth was harvested from the fermentors and centrifuged at 3000×g to remove the biomass. The supernatants were filtered using 0.22 μm SteriTop® filters. The filtered supernatants (filtrates) were stored at 5-10° C. The protein concentration was determined using a Microplate BCA™ Protein Assay Kit in which bovine serum albumin was used as a protein standard. The composition of the filtrates was supplemented by replacement of the filtrate protein with purified beta-glucosidase of SEQ ID NO: 10, GH10 xylanase of SEQ ID NO: 12, and beta-xylosidase of SEQ ID NO: 14 at 5%, 5%, and 3% of total protein, respectively, which resulted in mixtures 9, 10, 11, 12, and 13.

### Example 6

### Activity Assays on Pretreated Corn Stover

The activities of the fermentation broth filtrates 1-8 were measured for their ability to hydrolyze pretreated corn cobs and stover (PCCS) to produce sugars or for their ability to hydrolyze cellulose measured by reduced fluorescence using a fluorescence cellulose decay (FCD) assay (WO 2011/008785).

A pretreated biomass mixture consisting of dilute acid pretreated corn stover and corn cobs (PCCS) was diluted with water and adjusted to pH 5.0 prior to addition of 0.1 ml of fermentation broth filtrates 1-8 from Examples 1 and 2 plus 0.5 mg of purified beta-glucosidase of SEQ ID NO: 10, 0.5 mg of purified GH10 xylanase of SEQ ID NO: 12, and 0.3 mg of purified beta-xylosidase of SEQ ID NO: 14. The final composition was 20 g total weight with approximately 17% dry weight solids from biomass. The resulting enzyme/biomass slurry was incubated with constant mixing at 12 rpm for 5 days at 50° C. prior to measurement of the enzyme activity by measurement of resulting glucose after filtration of the hydrolysate slurry by centrifugation on a 96-well MULTISCREEN® HV 0.45 μm membrane plate (Millipore) at 3000 rpm for 10 minutes using a SORVALL® RT7 plate centrifuge (Thermo Fisher Scientific). When not used immediately, filtered sugary aliquots were frozen at −20° C. Sugar concentrations of samples diluted in 0.005 M H₂SO₄ were measured after elution by 0.005 M H₂SO₄ at a flow rate of 0.6 ml per minute from a 4.6×250 mm AMINEX® HPX-87H column (Bio-Rad Laboratories, Inc.) at 65° C. with quantitation by integration of the glucose signal from refractive index detection using a CHEMSTATION® AGILENT® 1100 HPLC (Agilent Technologies) calibrated by pure sugar samples (Absolute Standards).

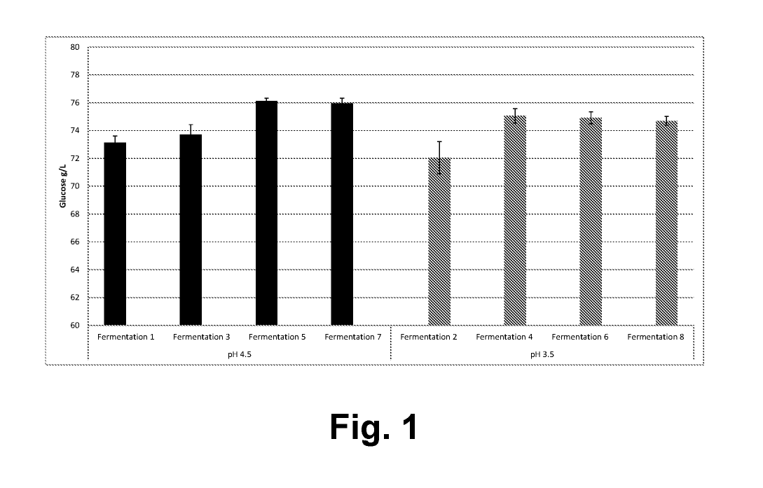

The results of the PCCS hydrolysis reactions in the 20 g assays are shown in FIG. 1. Fermentation broth filtrates 1 and 2 lack catalase. Although all of the fermentation broth filtrates were added at the same volumetric dose (0.1 ml of filtered fermentation broth) and supplemented with the same amount of purified beta-glucosidase of SEQ ID NO: 10, GH10 xylanase of SEQ ID NO: 12, and beta-xylosidase of SEQ ID NO: 14, the results demonstrated that enzyme compositions that are the result of co-cultures that produce catalase have higher yields of glucose as a result of having higher hydrolytic activity per volume, or more activity per production unit. This improvement in glucose is approximately 4% when fermenting at pH 4.5 with 10% or 25% seed co-culture, and approximately 4% when fermenting at pH 3.5 with 5%, 10% or 25% seed co-culture.

Measurement of the activity of mixtures 1-8 (Examples 1 and 2) was achieved by addition of appropriate enzyme dilution into slurries of biomass, incubation for 24 to 144 hours at 50° C., and measurement of the resulting drop in fluorescent signal caused by cellulose hydrolysis that results from the reduced binding of Calcofluor (FB-28, Sigma) to cellulose according to Wischmann et al., 2012, Methods Enzymol. 510: 19-36.

The PCCS described above was further modified by 6 hours of wet grinding in a COSMOS wet grinder (EssEmm Corp), sieved through a 425 μm mesh with an AS 200 Vibratory Sieve (Retsch), diluted with water, buffered with 60 mM acetate, 180 μM FB-28, pH adjusted, and autoclaved at 121° C. for 45 minutes to produce a material that was 6.25% total dry weight solids, pH 5.0. The substrate is referred to as FCD-GS-PCCS; 200 μl of FCD-GS-PCCS were placed in Costar 3364 plates (Corning).

Mixtures 1-8 were diluted 25× v/v and then serially diluted two-fold in milliQ water in 96 well deep well plates (Axygen), resulting in 8 enzyme dilutions from 25× v/v to 3200× v/v for each mixture. Fifty μl of each dilution of the mixtures from the plates were then added to each corresponding well of the plate containing FCD-GS-PCCS, equivalent of approximately 2 μl to 0.04 μl of original fermentations. The plates were heat sealed using an ALPS 300™ automated lab plate sealer (ABgene Inc.). The reaction mixtures were mixed by inverting and shaking the 96-well plate at the beginning of hydrolysis and before taking each sample time point. Final PCCS concentration was 50 g per liter in 50 mM sodium acetate pH 5.0, with 150 μM FB-28. PCCS hydrolysis was performed with incubation at 50° C. and 55° C. without additional stirring except during sampling as described. Each reaction was performed in triplicate, and plotted values were the averages of replicates. The fluorescence of no-enzyme and high enzyme controls (>5 times half maximal digestion) were used to determine 0% (Fmin) and 100% (Fmax) conversion. The conversion for any dose was calculated from the measured fluorescence (Fsample) with excitation at 365 and emission at 465 as follows:

conversion %=(Fmax−Fsample)/(Fmax−Fmin). (Equation 1)

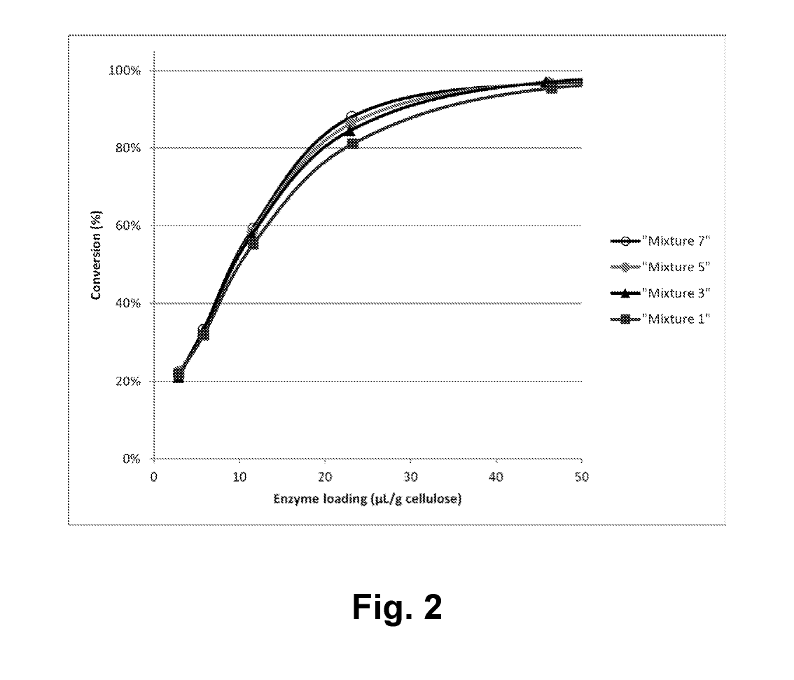

FIG. 2 shows the dose response plot for mixtures 1, 3, 5 and 7 (pH 4.5 fermentation) at 50° C. and pH 5.0 for 6 days, which demonstrates that increasing the percentage of the catalase-expressing seed in co-culture yielded higher cellulose hydrolysis. Since cellulose hydrolysis is correlated with the enzymatic release of glucose, the results demonstrate that higher catalase expression correlates with more glucose release (See Wischmann et al., 2012, supra), when dosing equal volume of fermentation broth filtrate.

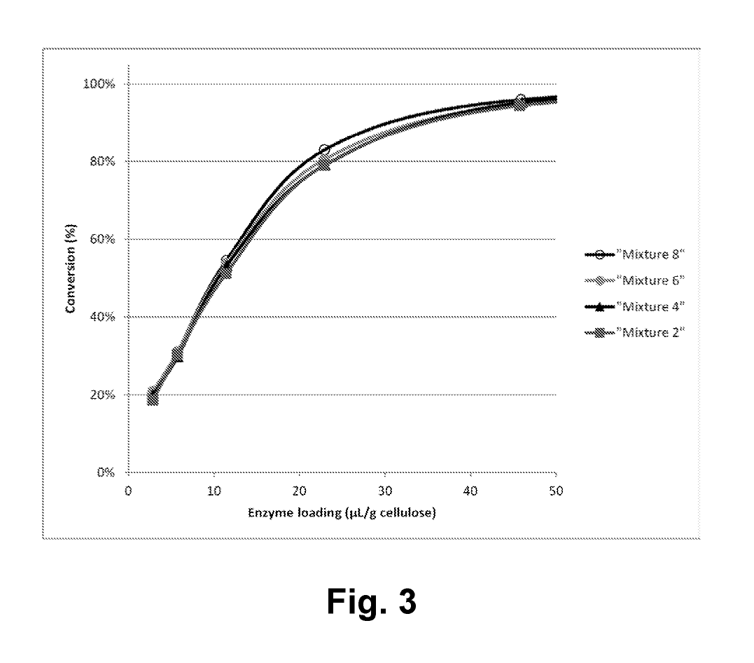

FIG. 3 shows the dose response plot for mixtures 2, 4, 6 and 8 (pH 3.5 fermentation) at 50° C. and pH 5.0 for 6 days, with demonstration that increasing the percentage of the catalase-expressing seed in co-culture yielded higher cellulose hydrolysis. Since cellulose hydrolysis is correlated with the enzymatic release of glucose, the results demonstrated that higher catalase expression correlates with more glucose release (See Wischmann et al., 2012, supra), when dosing equal volume of fermentation broth filtrate.

### Example 7

### Storage Stability of Co-Fermentation Broths

Fermentation broths 1-8 described in Examples 1 and 2 were sterile filtered, aliquoted into sterile 96-well deep-well plates (Axygen), sealed using an ALPS 300™ automated lab plate sealer (ABgene Inc.), and stored for 4 weeks under aseptic conditions at 4, 25, 40 and 50° C. The resulting samples were supplemented into mixtures equivalent to mixtures 1 through 8 with beta-glucosidase, GH10 xylanase, and beta-xylosidase as described in Examples 1 and 2, and assayed using the FCD assay described in Example 6, with incubation for 7 days.

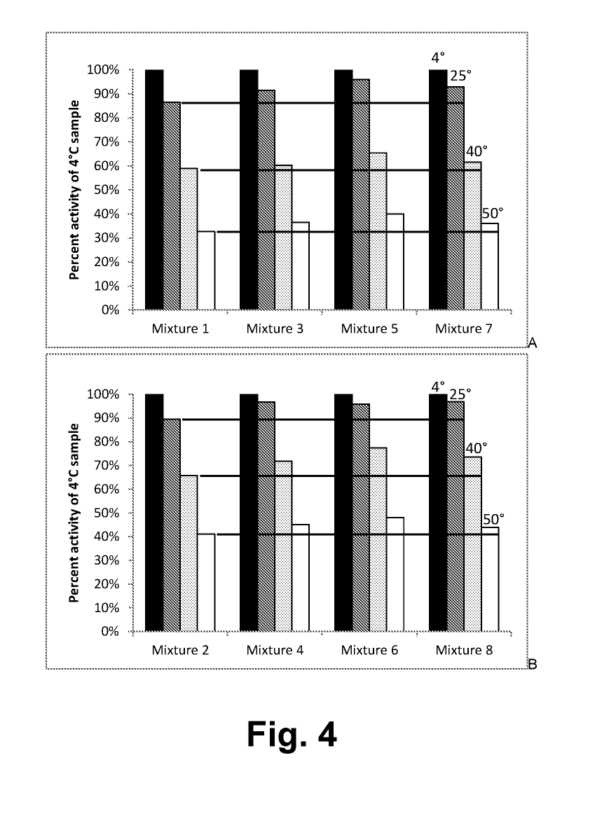

FIG. 4A shows the conversion achieved for mixtures 1, 3, 5, and 7 (pH 4.5 fermentation) as compared by ratio with the value attained by samples stored at 4° C. (100% of 4° C. sample) for each of the storage temperatures. Mixture 1 was produced from Fermentation 1, which has no co-culture seed strain expressing catalase. All catalase-containing mixtures 3, 5, and 7 show higher stabilities (retention of activity) than mixture 1 after storage at elevated temperatures. FIG. 4B shows the conversion achieved for mixtures 4, 6 and 8 (pH 3.5 fermentation) as compared by ratio with the value attained by samples stored at 4° C. (100% of 4° C. sample) for each of the storage temperatures. Mixture 2 was produced from Fermentation 2, which has no co-culture seed strain containing expressing catalase. All catalase-containing mixtures 4, 6 and 8 show higher stabilities (retention of activity) than mixture 2 after storage at elevated temperatures. Specifically, catalase-expressing co-culture broths show 5% to 9% higher stability at 25° C., 1% to 12% higher stability at 40° C. storage, and 3% to 7% higher stability at 50° C. storage than the control mixtures.

### Example 8

### Storage Stability of Broths with Bolus Catalase Addition into Fermentation

The filtered fermentation broths described in Example 5 were stored for 4 weeks under aseptic conditions at 4, 25, and 40° C. as described in Example 7 and then supplemented equivalently to mixtures 9, 10, 11, and 12 from Example 5 with purified beta-glucosidase of SEQ ID NO: 10, GH10 xylanase of SEQ ID NO: 12, and beta-xylosidase of SEQ ID NO: 14 as described previously. The hydrolysis activities of these mixtures in serial dilution were measured as described in Example 6, with incubation at 55° C. for 5 days generating a hydrolysis profile similar to that shown in FIGS. 2 and 3.

A curve approximating the hydrolysis profile was generated based on the equation

$$conversion \% = \frac{ConversionMax \% \cdot {( \frac{X}{K} )}^{P}}{1 + {( \frac{X}{K} )}^{P}} \qquad ( Equation 2 )$$

where the constants P (power function) and K (half-max of hydrolysis) for each sample dilution curve is optimized by the Excel plug-in Solver (Microsoft) to minimize the sum of square of errors to fit from the enzyme loadings X (in mg protein from broth, or in u of broth) and calculated conversion %. These constants can then be used to interpolate the enzyme loading necessary to reach a desired target (T) of conversion, e.g., 80% conversion:

$$Enzymeloading = K \cdot {( \frac{T}{ConversionMax \% - T} )}^{\frac{1}{P}} \qquad ( Equation 3 )$$

Calculation of the enzyme loading to reach a constant hydrolysis percent as target (T) allows for the comparison of efficiency of different enzyme samples e.g., the μl of fermentation broth/g cellulose necessary to reach 80% conversion.

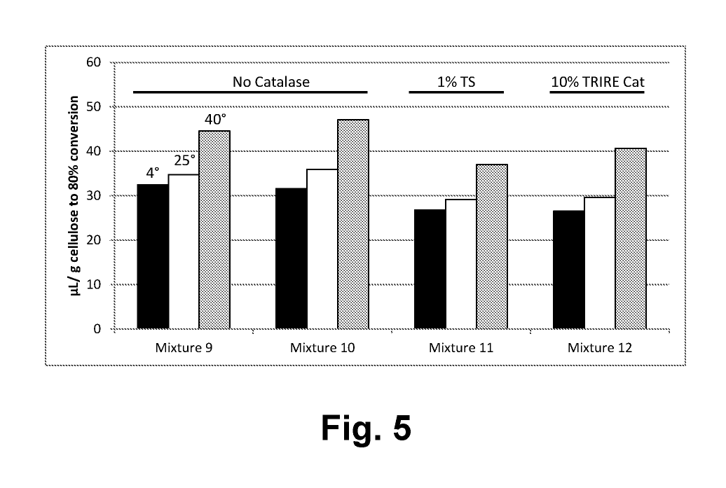

FIG. 5 shows the benefit of catalase protein added either derived from Example 3 or Example 4 to the storage performance of Fermentation broths 11 and 12, in that at all temperatures of stored material, Mixtures 11 and 12 with catalase addition into fermentation outperformed mixtures 9 (pH 3.5) and 10 (pH 4.5) that lack catalase, by requiring fewer μl to reach the target 80% conversion. This improvement in storage performance resulted in a 15% to 18% reduction in μl required after 4° C. and 25° C. storage, and a 9% to 15% reduction in μl required after 40° C. storage.

### Example 9

### Effect of Addition of Terminox® Supreme to Mixture 13 after Fermentation

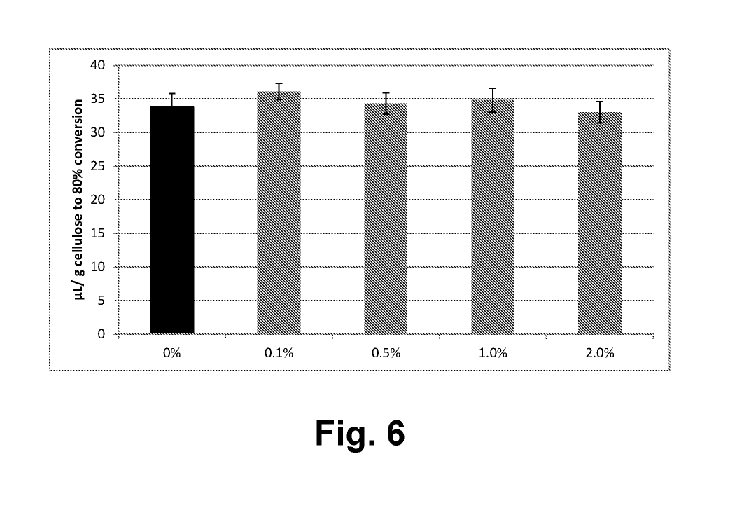

Filtered fermentation broth 13 from Example 5 of Strain C, a Trichoderma strain not over-expressing catalase, was measured as in prior Examples for protein content, and mixtures were made by supplementation by replacement of broth protein by purified beta-glucosidase of SEQ ID NO: 10, GH10 xylanase of SEQ ID NO: 12, and beta-xylosidase of SEQ ID NO: 14 at 5%, 5% and 3% respectively, and with replacement by Terminox® Supreme used as is, measured as 13.5 mg per ml using a Microplate BCA™ Protein Assay Kit in which bovine serum albumin was used as a protein standard, to final mixtures with Terminox® Supreme protein at 0%, 0.1%, 0.5%, 1% and 2% w/w protein. The activity of mixture 13 in hydrolysis was measured by FCD, as described in Example 6, at pH 5 and 55° C. for 5 days, and the μl/g cellulose loading necessary to reach 80% conversion was calculated by interpolation of the fitted curve as in Example 8. FIG. 6 shows that the addition of Terminox® Supreme, a source of catalase, after fermentation did not improve the performance significantly (the best mixture, with 2% Terminox® Supreme protein, was 2% better than the 0% Terminox® Supreme mixture, but with standard deviation of 3-6%). This benefit was not nearly as much as was observed when the catalase was added during fermentation as in mixture 11 or 12 in FIG. 5, Example 8.

### Example 10

### Western Blots of Co-Culture

Antibody was raised in rabbits as a polyclonal response against the synthetic peptide KQAFGDTDDFSKHG (SEQ ID NO: 15), representing a portion of the sequence of the cellobiohydrolase I of SEQ ID NO: 2 (residues 371-384). The antibody is referred to as αCBH1 primary antibody.

Filtered fermentation broths 1-8 from Examples 1 and 2 were diluted to approximately 1 μg protein in 5 μl of water, then were further diluted 1:1 with 2× Laemlli buffer (Bio-Rad Laboratories, Inc.) with 1× TCEP (Thermo Scientific) and heated at 95° C. for 5 minutes, cooled, centrifuged, and loaded onto a 26-well 10% Criterion® TGX StainFree SDS-PAGE gel (Bio-Rad Laboratories, Inc.). The gel was run at 300 volts for 20 minutes. The gel was transferred onto an Immune-Blot PVDF membrane (Bio-Rad Laboratories, Inc.) using semi-dry Trans-Blot® Turbo™ Blotting System (Bio-Rad Laboratories, Inc.). The membrane was washed twice for 5 minutes in Tris buffer saline pH 7.5 (TBS; 20 mM Tris-500 mM NaCl) on a rocker at room temperature and incubated with 1% BSA Blocking Buffer in TBST (TBS+0.05% TWEEN® 20) for 1 hour. All subsequent steps included three washing steps for 5 minutes with TBST. The blot was incubated for 1 hour with αCBH1 primary antibody (Covance) diluted 1/10,000 with TBST, followed by a 1 hour incubation with secondary antibody goat anti-rabbit HRP (Jackson ImmunoResearch Laboratories) diluted 1/10,000 TBST. The Western Blot had a final wash in TBS with SuperSignal West Pico Substrate (Thermo Scientific) before detection using Chemi-Luminescence setting for Blots on a ChemiDoc MP (Bio-Rad Laboratories, Inc.). Quantitation of the blot intensity was by the default settings for ImageLab (Bio-Rad Laboratories, Inc.).

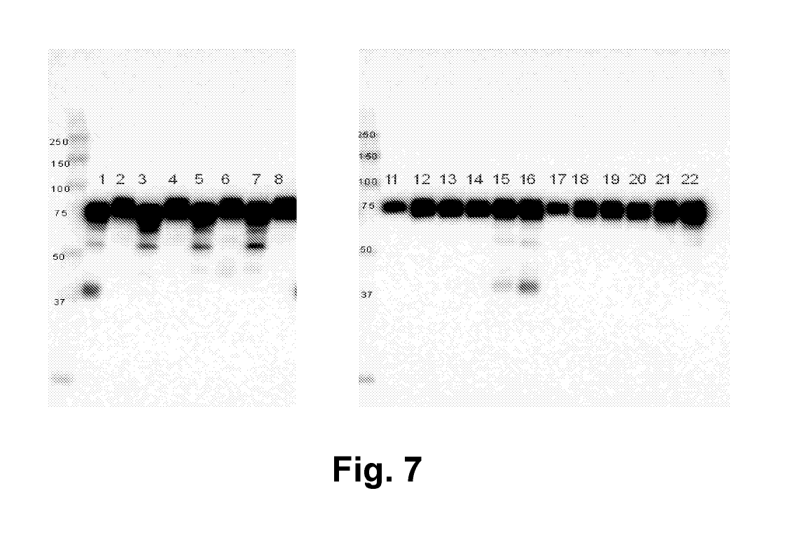

FIG. 7 shows the resulting Western blot image, with lanes 1-8 representing the filtered fermentation broths 1-8, produced according to Examples 1 and 2, as co-cultured with catalase-expressing strains as summarized in Table 1. A band of approximately 37,000 daltons represents a fragmentation of the cellobiohydrolase I that occurred in samples with AA9 polypeptide expression but without catalase expression when fermented at pH 4.5. The co-culture samples expressing catalase (lanes 3-8) do not show this band. Lanes 11-16 represent BCA Microplate assay protein-normalized (1 μg) loadings of daily samples from days 2 to 7, respectively, for fermentation 1 (0% catalase over-expression seed B), while lanes 17-22 represent the equivalent samples for fermentation 5 (10% catalase over-expression seed B). The development of the fragment at approximately 37,000 daltons was visible in the fermentation without catalase co-culture, while the fragment was absent in a co-culture with 10% seed from catalase-producing strain B, demonstrating that the fragmentation occurs during fermentation, and catalase expression reduces this fragmentation to levels not visible to the eye.

### Example 11

### Western Blots of Catalase Protein Addition During Fermentation

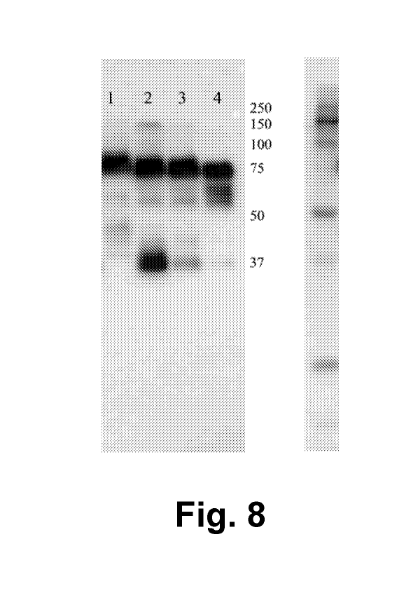

Approximately 1 μg of broth protein in 5 μl of water from Example 5 fermentation broth filtrates (fermentations 9 through 12, see Table 3, representing lanes 1 through 4, respectively in FIG. 8) were treated as described in Example 10. FIG. 8 shows high amounts of the 37,000 dalton fragment from fermentation 10, shown in lane 2. Addition of catalase protein with seed at the start of fermentation (fermentations 11 and 12) showed greatly reduced amount of 37,000 dalton fragment in lanes 3 and 4, respectively, compared with lane 2 where catalase protein was not added with seed, illustrating the higher integrity of this protein after fermentations with catalase. Lane 1 shows fermentation 9, grown at pH 3.5, where lesser amounts of the 37,000 dalton fragment were seen.

The present invention is further described by the following numbered paragraphs:

Paragraph [1]: A method of inhibiting AA9 lytic polysaccharide monooxygenase catalyzed inactivation of an enzyme composition or a component thereof, said method comprising: adding one or more oxidoreductases selected from the group consisting of a catalase, a laccase, a peroxidase, and a superoxide dismutase to the enzyme composition comprising an AA9 lytic polysaccharide monooxygenase and one or more enzyme components, wherein the one or more added oxidoreductases inhibit AA9 lytic polysaccharide monooxygenase catalyzed inactivation of the one or more enzyme components of the enzyme composition.

Paragraph [2]: The method of paragraph 1, wherein the one or more oxidoreductases is a catalase.

Paragraph [3]: The method of paragraph 1, wherein the one or more oxidoreductases is a laccase.

Paragraph [4]: The method of paragraph 1, wherein the one or more oxidoreductases is a peroxidase.

Paragraph [5]: The method of paragraph 1, wherein the one or more oxidoreductases is a superoxide dismutase.

Paragraph [6]: The method of paragraph 1, wherein the one or more oxidoreductases is a combination of two or more oxidoreductases selected from the group consisting of a catalase, a laccases, a peroxidase, and a superoxide dismutase.

Paragraph [7]: The method of any one of paragraphs 1-6, wherein the enzyme composition comprises one or more components selected from the group consisting of a hydrolase, an isomerase, a ligase, a lyase, an oxidoreductase, or a transferase.

Paragraph [8]: The method of any one of paragraphs 1-6, wherein the enzyme composition comprises one or more components selected from the group consisting of a cellulase, an AA9 polypeptide, a hemicellulase, a cellulose inducing protein, an esterase, an expansin, a ligninolytic enzyme, a pectinase, a protease, and a swollenin.

Paragraph [9]: The method of paragraph 8, wherein the cellulase is one or more enzymes selected from the group consisting of an endoglucanase, a cellobiohydrolase, and a beta-glucosidase.

Paragraph [10]: The method of paragraph 8, wherein the hemicellulase is one or more enzymes selected from the group consisting of a xylanase, an acetylxylan esterase, a feruloyl esterase, an arabinofuranosidase, a xylosidase, and a glucuronidase.

Paragraph [11]: The method of any one of paragraphs 1-10, wherein the protein ratio of the added oxidoreductase to the AA9 lytic polysaccharide monooxygenase is in the range of about 1:250 to about 1:10, e.g., about 1:200 to about 1:10, about 1:150 to about 1:15, about 1:100 to about 1:15, about 1:75 to about 1:20, or about 1:50 to about 1:25.

Paragraph [12]: The method of any one of paragraphs 1-11, wherein the amount of inhibition of the AA9 lytic polysaccharide monooxygenase catalyzed inactivation is higher in the presence of the one or more added oxidoreductases compared to the absence of the one or more added oxidoreductases.

Paragraph [13]: A method for increasing production of an enzyme composition, said method comprising: (a) fermenting a host cell to produce the enzyme composition in the presence of one or more added oxidoreductases selected from the group consisting of a catalase, a laccases, a peroxidase, and a superoxide dismutase, wherein the enzyme composition comprises an AA9 lytic polysaccharide monooxygenase and one or more enzyme components, wherein the one or more added oxidoreductases inhibit the AA9 lytic polysaccharide monooxygenase catalyzed inactivation of the one or more enzyme components of the enzyme composition, and wherein the amount of the enzyme composition produced in the presence of the one or more added oxidoreductases is higher compared to the amount of the enzyme composition produced in the absence of the added one or more oxidoreductases; and optionally (brecovering the enzyme composition.

Paragraph [14]: The method of paragraph 13, wherein the one or more added oxidoreductases is a catalase.

Paragraph [15]: The method of paragraph 13, wherein the one or more added oxidoreductases is a laccase.

Paragraph [16]: The method of paragraph 13, wherein the one or more added oxidoreductases is a peroxidase.

Paragraph [17]: The method of paragraph 13, wherein the one or more added oxidoreductases is a superoxide dismutase.

Paragraph [18]: The method of paragraph 13, wherein the one or more added oxidoreductases is a combination of two or more oxidoreductases selected from the group consisting of a catalase, a laccases, a peroxidase, and a superoxide dismutase.

Paragraph [19]: The method of any one of paragraphs 13-18, wherein the host cell comprises an AA9 lytic polysaccharide monooxygenase native to the host cell.

Paragraph [20]: The method of any one of paragraphs 13-18, wherein the host cell comprises an AA9 lytic polysaccharide monooxygenase heterologous to the host cell.

Paragraph [21]: The method of any one of paragraphs 13-18, wherein the host cell comprises an AA9 lytic polysaccharide monooxygenase native to the host cell and an AA9 lytic polysaccharide monooxygenase heterologous to the host cell.

Paragraph [22]: The method of paragraph any one of paragraphs 13-21, wherein the enzyme composition comprises one or more components selected from the group consisting of a hydrolase, an isomerase, a ligase, a lyase, an oxidoreductase, or a transferase.

Paragraph [23]: The method of any one of paragraphs 13-21, wherein the enzyme composition comprises one or more components selected from the group consisting of a cellulase, an AA9 polypeptide, a hemicellulase, a cellulose inducing protein, an esterase, an expansin, a ligninolytic enzyme, a pectinase, a protease, and a swollenin.

Paragraph [24]: The method of paragraph 23, wherein the cellulase is one or more enzymes selected from the group consisting of an endoglucanase, a cellobiohydrolase, and a beta-glucosidase.

Paragraph [25]: The method of paragraph 23, wherein the hemicellulase is one or more enzymes selected from the group consisting of a xylanase, an acetylxylan esterase, a feruloyl esterase, an arabinofuranosidase, a xylosidase, and a glucuronidase.

Paragraph [26]: The method of any one of paragraphs 13-25, wherein the one or more added oxidoreductases are added to the fermentation.

Paragraph [27]: The method of any one of paragraphs 13-25, wherein the one or more added oxidoreductases are recombinantly produced by the host cell.

Paragraph [28]: The method of any one of paragraphs 13-25, wherein the one or more added oxidoreductases are recombinantly produced by co-culture of the recombinant cell with a second host cell.

Paragraph [29]: The method of any one of paragraphs 13-25, wherein the one or more added oxidoreductases are added to the fermentation and recombinantly produced by the host cell.

Paragraph [30]: The method of any one of paragraphs 13-25, wherein the one or more added oxidoreductases are added to the fermentation and recombinantly produced by co-culture of the recombinant cell with a second host cell.

Paragraph [31]: The method of any one of paragraphs 13-25, wherein the one or more added oxidoreductases are recombinantly produced by the host cell and recombinantly produced by co-culture of the recombinant cell with a second host cell.

Paragraph [32]: The method of any one of paragraphs 13-25, wherein the one or more added oxidoreductases are added to the fermentation, recombinantly produced by the host cell, and recombinantly produced by co-culture of the recombinant cell with a second host cell.

Paragraph [33]: The method of any one of paragraphs 13-32, wherein the protein ratio of the added oxidoreductase to the AA9 lytic polysaccharide monooxygenase is in the range of about 1:250 to about 1:10, e.g., about 1:200 to about 1:10, about 1:150 to about 1:15, about 1:100 to about 1:15, about 1:75 to about 1:20, or about 1:50 to about 1:25.

Paragraph [34]: The method of any one of paragraphs 13-33, wherein the inhibition of the AA9 lytic polysaccharide monooxygenase catalyzed inactivation is higher in the presence of the one or more added oxidoreductases compared to the absence of the one or more added oxidoreductases.

Paragraph [35]: A method for stabilizing an enzyme composition, comprising adding one or more oxidoreductases selected from the group consisting of a catalase, a laccases, a peroxidase, and a superoxide dismutase to the enzyme composition, wherein the enzyme composition comprises an AA9 lytic polysaccharide monooxygenase and one or more enzyme components, and wherein the one or more added oxidoreductases inhibit AA9 lytic polysaccharide monooxygenase catalyzed inactivation of the one or more enzyme components of the enzyme composition.

Paragraph [36]: The method of paragraph 35, wherein the one or more oxidoreductases is a catalase.

Paragraph [37]: The method of paragraph 35, wherein the one or more oxidoreductases is a laccase.

Paragraph [38]: The method of paragraph 35, wherein the one or more oxidoreductases is a peroxidase.

Paragraph [39]: The method of paragraph 35, wherein the one or more oxidoreductases is a superoxide dismutase.

Paragraph [40]: The method of paragraph 35, wherein the one or more oxidoreductases is a combination of two or more oxidoreductases selected from the group consisting of a catalase, a laccases, a peroxidase, and a superoxide dismutase.

Paragraph [41]: The method of any one of paragraphs 35-40, wherein the enzyme composition comprises one or more components selected from the group consisting of a hydrolase, an isomerase, a ligase, a lyase, an oxidoreductase, or a transferase.

Paragraph [42]: The method of any one of paragraphs 35-40, wherein the enzyme composition comprises one or more components selected from the group consisting of a cellulase, an AA9 polypeptide, a hemicellulase, a cellulose inducing protein, an esterase, an expansin, a ligninolytic enzyme, a pectinase, a protease, and a swollenin.

Paragraph [43]: The method of paragraph 42, wherein the cellulase is one or more enzymes selected from the group consisting of an endoglucanase, a cellobiohydrolase, and a beta-glucosidase.

Paragraph [44]: The method of paragraph 42, wherein the hemicellulase is one or more enzymes selected from the group consisting of a xylanase, an acetylxylan esterase, a feruloyl esterase, an arabinofuranosidase, a xylosidase, and a glucuronidase.

Paragraph [45]: The method of any one of paragraphs 35-44, wherein the protein ratio of the added oxidoreductase to the AA9 lytic polysaccharide monooxygenase is in the range of about 1:250 to about 1:10, e.g., about 1:200 to about 1:10, about 1:150 to about 1:15, about 1:100 to about 1:15, about 1:75 to about 1:20, or about 1:50 to about 1:25.

Paragraph [46]: The method of any one of paragraphs 35-45, wherein the amount of inhibition of the AA9 lytic polysaccharide monooxygenase catalyzed inactivation is higher in the presence of the one or more added oxidoreductases compared to the absence of the one or more added oxidoreductases.

Paragraph [47]: A composition comprising an AA9 lytic polysaccharide monooxygenase and one or more added oxidoreductases selected from the group consisting of a catalase, a laccases, a peroxidase, and a superoxide dismutase, wherein the protein ratio of the added oxidoreductase to the AA9 lytic polysaccharide monooxygenase is in the range of about 1:250 to about 1:10, e.g., about 1:200 to about 1:10, about 1:150 to about 1:15, about 1:100 to about 1:15, about 1:75 to about 1:20, or about 1:50 to about 1:25.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A method of inhibiting AA9 lytic polysaccharide monooxygenase catalyzed inactivation of an enzyme composition or a component thereof, said method comprising: adding one or more oxidoreductases selected from the group consisting of a catalase, a laccase, a peroxidase, and a superoxide dismutase to the enzyme composition comprising an AA9 lytic polysaccharide monooxygenase and one or more enzyme components, wherein the one or more added oxidoreductases inhibit AA9 lytic polysaccharide monooxygenase catalyzed inactivation of the one or more enzyme components of the enzyme composition.

2. The method of claim 1, wherein the enzyme composition further comprises one or more components selected from the group consisting of a hydrolase, an isomerase, a ligase, a lyase, an oxidoreductase, or a transferase.

3. The method of claim 1, wherein the enzyme composition further comprises one or more components selected from the group consisting of a cellulase, an AA9 polypeptide, a hemicellulase, a cellulose inducing protein, an esterase, an expansin, a ligninolytic enzyme, a pectinase, a protease, and a swollenin.

4. The method of claim 1, wherein the protein ratio of the added oxidoreductase to the AA9 lytic polysaccharide monooxygenase is in the range of about 1:250 to about 1:10.

5. The method of claim 1, wherein the amount of inhibition of the AA9 lytic polysaccharide monooxygenase catalyzed inactivation is higher in the presence of the one or more added oxidoreductases compared to the absence of the one or more added oxidoreductases.

6. A method for increasing production of an enzyme composition, said method comprising:
(a) fermenting a host cell to produce the enzyme composition in the presence of one or more added oxidoreductases selected from the group consisting of a catalase, a laccases, a peroxidase, and a superoxide dismutase, wherein the enzyme composition comprises an AA9 lytic polysaccharide monooxygenase and one or more enzyme components, wherein the one or more added oxidoreductases inhibit the AA9 lytic polysaccharide monooxygenase catalyzed inactivation of the one or more enzyme components of the enzyme composition, and wherein the amount of the enzyme composition produced in the presence of the one or more added oxidoreductases is higher compared to the amount of the enzyme composition produced in the absence of the added one or more oxidoreductases; and
(b) recovering the enzyme composition.

7. The method of claim 6, wherein the host cell comprises an AA9 lytic polysaccharide monooxygenase native to the host cell; an AA9 lytic polysaccharide monooxygenase heterologous to the host cell; or an AA9 lytic polysaccharide monooxygenase native to the host cell and an AA9 lytic polysaccharide monooxygenase heterologous to the host cell.

8. The method of claim 6, wherein the enzyme composition further comprises one or more components selected from the group consisting of a hydrolase, an isomerase, a ligase, a lyase, an oxidoreductase, or a transferase.

9. The method of claim 6, wherein the enzyme composition further comprises one or more components selected from the group consisting of a cellulase, an AA9 polypeptide, a hemicellulase, a cellulose inducing protein, an esterase, an expansin, a ligninolytic enzyme, a pectinase, a protease, and a swollenin.

10. The method of claim 6, wherein the one or more added oxidoreductases are added to the fermentation; the one or more added oxidoreductases are recombinantly produced by the host cell; the one or more added oxidoreductases are recombinantly produced by co-culture of the recombinant cell with a second host cell; the one or more added oxidoreductases are added to the fermentation and recombinantly produced by the host cell; the one or more added oxidoreductases are added to the fermentation and recombinantly produced by co-culture of the recombinant cell with a second host cell; the one or more added oxidoreductases are recombinantly produced by the host cell and recombinantly produced by co-culture of the recombinant cell with a second host cell; or the one or more added oxidoreductases are added to the fermentation, recombinantly produced by the host cell, and recombinantly produced by co-culture of the recombinant cell with a second host cell.

11. The method of claim 6, wherein the protein ratio of the added oxidoreductase to the AA9 lytic polysaccharide monooxygenase is in the range of about 1:250 to about 1:10.

12. The method of claim 6, wherein the inhibition of the AA9 lytic polysaccharide monooxygenase catalyzed inactivation is higher in the presence of the one or more added oxidoreductases compared to the absence of the one or more added oxidoreductases.

13. A method for stabilizing an enzyme composition, comprising adding one or more oxidoreductases selected from the group consisting of a catalase, a laccases, a peroxidase, and a superoxide dismutase to the enzyme composition, wherein the enzyme composition comprises an AA9 lytic polysaccharide monooxygenase and one or more enzyme components, and wherein the one or more added oxidoreductases inhibit AA9 lytic polysaccharide monooxygenase catalyzed inactivation of the one or more enzyme components of the enzyme composition.

14. The method of claim 13, wherein the enzyme composition further comprises one or more components selected from the group consisting of a hydrolase, an isomerase, a ligase, a lyase, an oxidoreductase, or a transferase.

15. The method of claim 13, wherein the enzyme composition further comprises one or more components selected from the group consisting of a cellulase, an AA9 polypeptide, a hemicellulase, a cellulose inducing protein, an esterase, an expansin, a ligninolytic enzyme, a pectinase, a protease, and a swollenin.

16. The method of claim 13, wherein the protein ratio of the added oxidoreductase to the AA9 lytic polysaccharide monooxygenase is in the range of about 1:250 to about 1:10.

17. The method of claim 13, wherein the amount of inhibition of the AA9 lytic polysaccharide monooxygenase catalyzed inactivation is higher in the presence of the one or more added oxidoreductases compared to the absence of the one or more added oxidoreductases.

18. (canceled)

19. (canceled)

20. (canceled)
